(19) United States
(12) Patent Application Publication
(10) Pub. No.: US 2012/0252810 A1
(43) Pub. Date: Oct. 4, 2012

(54) FATTY ACID NON-FLUSHING NIACIN DERIVATIVES AND THEIR USES

(75) Inventors: Chi B. Vu, Arlington, MA (US); Jean E. Bemis, Arlington, MA (US); Michael R. Jirousek, Cambridge, MA (US); Jill C. Milne, Brookline, MA (US); Amal Ting, Newton, MA (US)

(73) Assignee: CATABASIS PHARMACEUTICALS, INC., Cambridge, MA (US)

(21) Appl. No.: 13/439,168

(22) Filed: Apr. 4, 2012

Related U.S. Application Data
(60) Provisional application No. 61/471,462, filed on Apr. 4, 2011.

(51) Int. Cl. A61K 31/4406; C07D 401/06; A61K 31/4439; C07D 409/12; A61K 31/4436; C07D 413/06; A61K 31/5377; A61P 3/06; A61P 1/16; A61P 9/10; A61P 3/04; A61P 3/10; A61P 9/00; A61P 27/02; C07D 213/56

(52) U.S. Cl. 514/235.5; 546/336; 514/357; 546/335; 546/276.4; 514/343; 546/280.7; 514/342; 544/131

(57) ABSTRACT

The invention relates to fatty non-flushing acid niacin derivatives; compositions comprising an effective amount of a fatty acid non-flushing niacin derivative; and methods for treating or preventing an metabolic disease comprising the administration of an effective amount of a fatty acid non-flushing niacin derivative.

### PRIORITY

This application claims the benefit of U.S. Provisional Application No. 61/471,462 filed Apr. 4, 2011, the entire disclosure of which is relied on and incorporated into this application by reference.

### FIELD OF THE INVENTION

The invention relates to fatty acid non-flushing niacin derivatives; compositions comprising an effective amount of a fatty acid non-flushing niacin derivative; and methods for treating or preventing a metabolic disease comprising the administration of an effective amount of a fatty acid non-flushing niacin derivative. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, eicosapentaenoic acid (EPA) and docosahexaenoic acid (DHA) being the key marine derived omega-3 fatty acids. Both niacin and marine omega-3 fatty acids (EPA and DHA) have been shown to reduce cardiovascular disease, coronary heart disease, atherosclerosis and reduce mortality in patients with dyslipidemia, hypercholesterolemia, or Type 2 diabetes, and metabolic disease. Niacin at high dose (1.5 to 4 grams per day) has been shown to improve very low-density lipoprotein (“VLDL”) levels through lowering Apolipoprotein B (“ApoB”) and raising high density lipoprotein (“HDL”) through increasing Apolipoprotein A1 (“ApoA1”) in the liver. Niacin can also inhibit diacylglycerol acyltransferase-2, a key enzyme for TG synthesis (Kamanna, V. S.; Kashyap, M. L. Am. J. Cardiol. 2008, 101 (8A), 20B-26B). Unfortunately, niacin has many actions outside of the liver that detract from its therapeutic utility. The most common side effect of niacin is flushing, which can limit the dose a patient can tolerate. Flushing is thought to occur through the GPR109 receptor in the vasculature. More recently, non-flushing niacin derivatives of the following structures have been disclosed (Bachovchin, W. and Lai, H. “Non-flushing niacin analogues, and methods of use thereof” WO 2008/016968; Bachovchin et al, “Niacin mimetics, and methods of use thereof” WO 2011/163619). These derivatives can potentially have the same beneficial effect on raising HDL as niacin while displaying a lesser degree of flushing:

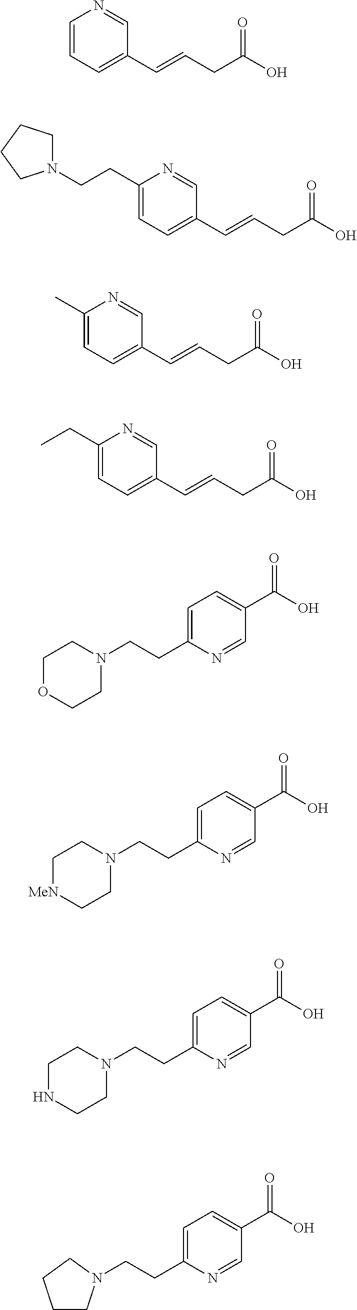

Omega-3 fatty acids (such as DHA and EPA) have been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been show to be improved in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in patients at risk of a cardiovascular event. Omega-3 fatty acids have also been taken as part of the dietary supplement portion of therapy used to treat dyslipidemia.

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42). Lipoic acid can potentially be useful in treating or preventing hypertriglyceridemia and diabetic dyslipidemia. Recent data suggested that the triglyceride-lowering effect of lipoic acid is due in part to its ability to stimulate triglyceride clearance and down-regulate liver triglyceride secretion, most likely via inhibition of DGAT-2 (Moreau et al, Archives of Biochemistry and Biophysics 2009, 485, p. 63-'71).

The ability to provide the effects of non-flushing niacin and omega-3 fatty acid in a synergistic way would provide a great benefit in treating the aforementioned diseases.

### SUMMARY OF THE INVENTION

The invention is based in part on the discovery of fatty acid non-flushing niacin derivatives and their demonstrated effects in achieving improved treatment that cannot be achieved by administering non-flushing niacin or fatty acids alone or in combination. These novel compounds are useful in the treatment or prevention of metabolic diseases including atherosclerosis, dyslipidemia, coronary heart disease, hypercholesterolemia, Type 2 diabetes, elevated cholesterol, metabolic syndrome and cardiovascular disease.

Accordingly in one aspect, a molecular conjugate is described which comprises a non-flushing niacin and a fatty acid covalently linked, wherein the fatty acid is selected from the group consisting of omega-3 fatty acids and fatty acids that are metabolized in vivo to omega-3 fatty acids, and the conjugate is capable of hydrolysis to produce free non-flushing niacin and free fatty acid.

In another aspect, compounds of the Formula I are described:

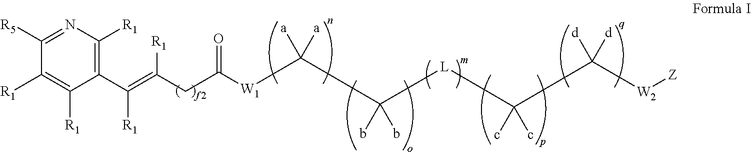

and pharmaceutically acceptable salts, hydrates, solvates, prodrugs, enantiomers and stereoisomers thereof;

wherein

each R₁ and R₂ is independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

R₅ is independently selected from the group consisting of H, -D, —Cl, —F, —CN, —NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₆ alkyl, —O—C₁-C₃ alkyl, —S(O)C₁-C₃ alkyl, —S(O)₂C₁-C₃ alkyl, an aryl, a cycloalkyl, a heterocycle and

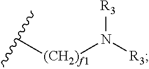

R₃ is independently H or C₁-C₆ alkyl, or both R₃ groups, when taken together with the nitrogen to which they are attached, can form

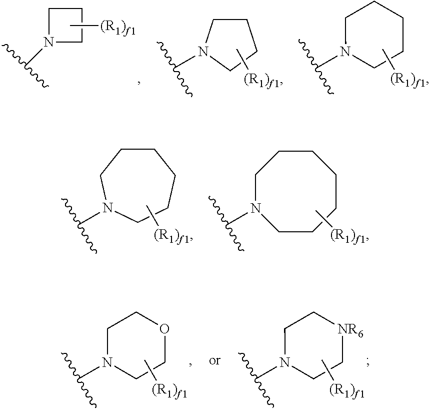

wherein f1 is 1, 2, 3 or 4; and f2 is 1, 2 or 3;

W₁ and W₂ are each independently null, O, S, NH, NR, or W₁ and W₂ can be taken together can form an imidazolidine or piperazine group, with the proviso that W₁ and W₂ can not be O simultaneously; each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

each L is independently-O—, —S—, —S(O)—, —S(O)₂—, —S—S—, —(C₁-C₆alkyl)-, —(C₃-C₆cycloalkyl)-, a heterocycle, a heteroaryl,

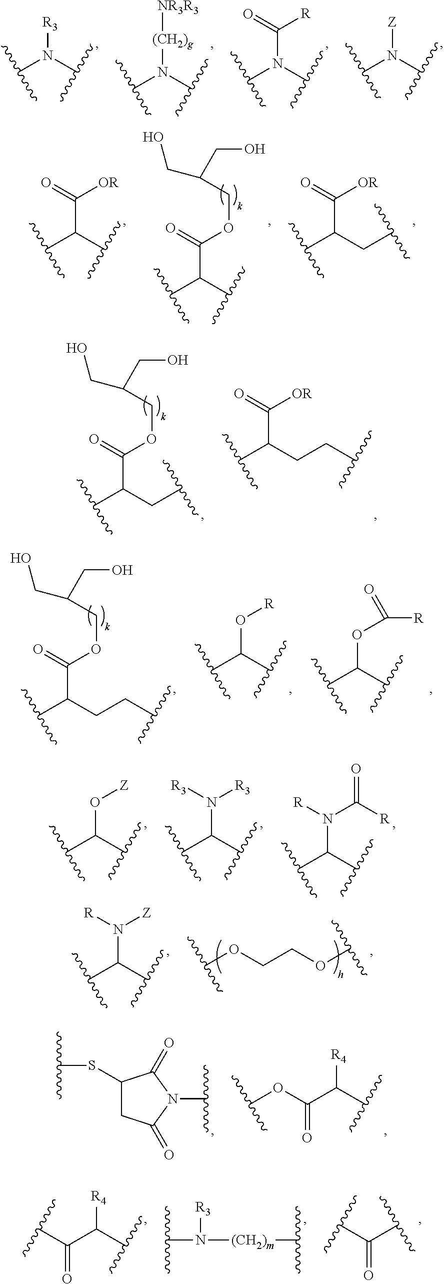

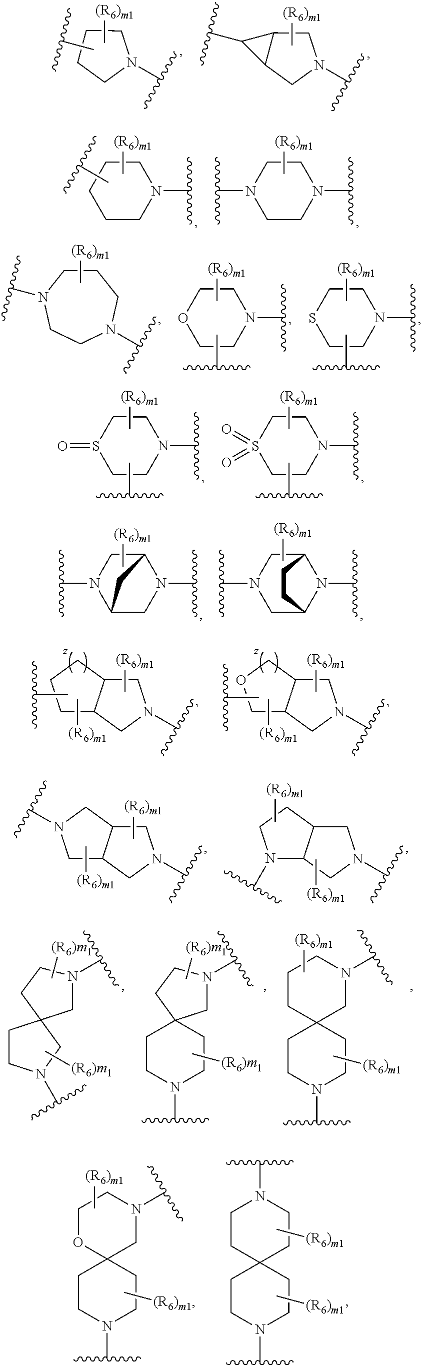

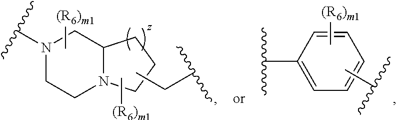

wherein the representation of L is not limited directionally left to right as is depicted, rather either the left side or the right side of L can be bound to the W₁ side of the compound of Formula I;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

each g is independently 2, 3 or 4;

each h is independently 1, 2, 3 or 4;

m is 0, 1, 2, 3, 4 or 5; if m is more than 1, then L can be the same or different; m1 is 0, 1, 2 or 3;

k is 0, 1, 2, or 3;

z is 1, 2, or 3;

each R₄ independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

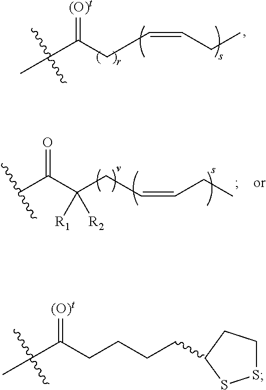

with the proviso that there is at least one

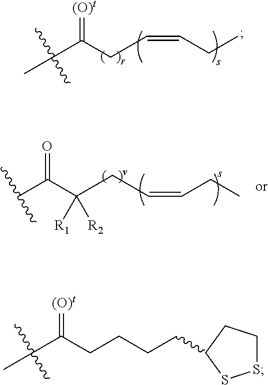

in the compound;

each r is independently 2, 3, or 7;

each s is independently 3, 5, or 6;

each t is independently 0 or 1;

each v is independently 1, 2, or 6;

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

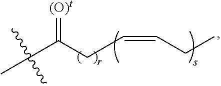

then t must be 0; and
when m, n, o, p, and q are each 0, and W₁ and W₂ are each null, then Z must not be

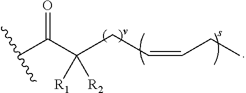

In another aspect, compounds of the Formula II are described:

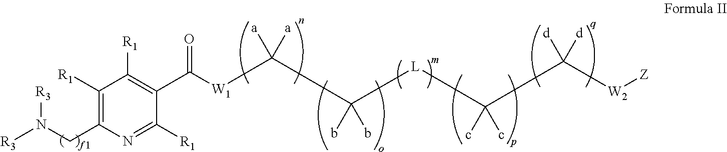

and pharmaceutically acceptable salts, hydrates, solvates, prodrugs, enantiomers and stereoisomers thereof;

wherein

each R₁ and R₂ is independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

R₃ is independently H or C₁-C₆ alkyl, or both R₃ groups, when taken together with the nitrogen to which they are attached, can form

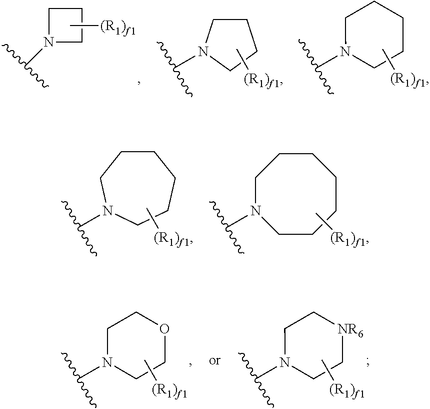

wherein f1 is 1, 2, 3 or 4;

W₁ and W₂ are each independently null, O, S, NH, NR, or W₁ and W₂ can be taken together can form an imidazolidine or piperazine group, with the proviso that W₁ and W₂ can not be O simultaneously;

each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

each L is independently-O—, —S—, —S(O)—, —S(O)₂—, —S—S—, —(C₁-C₆alkyl)-, —(C₃-C₆cycloalkyl)-, a heterocycle, a heteroaryl,

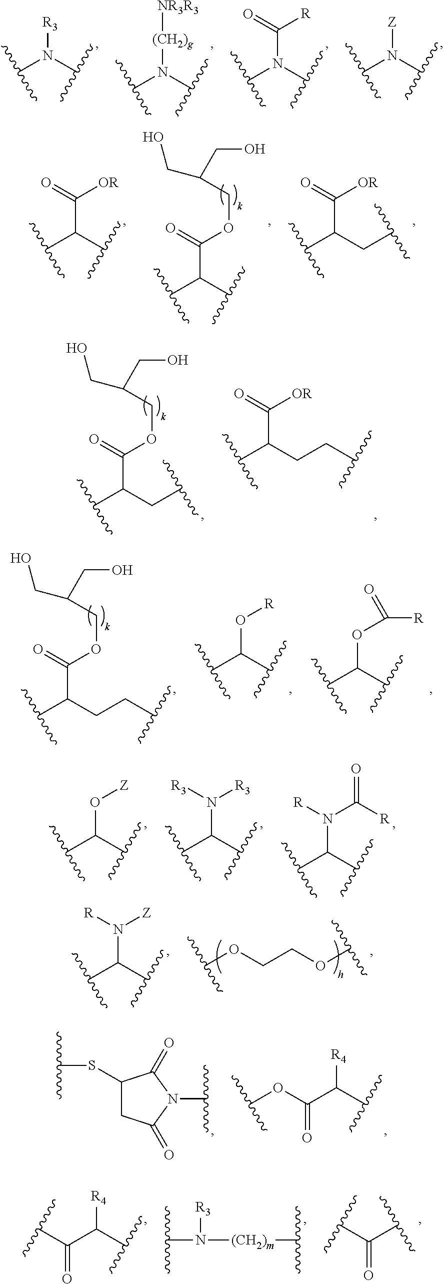

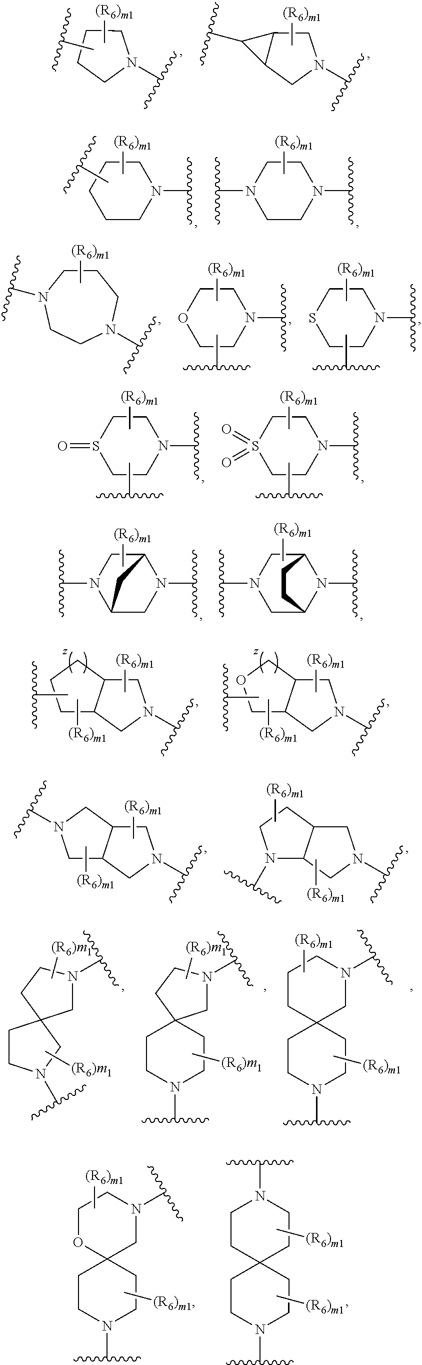

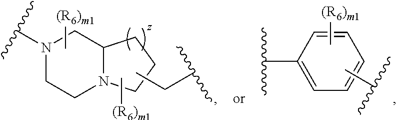

wherein the representation of L is not limited directionally left to right as is depicted, rather either the left side or the right side of L can be bound to the W₁ side of the compound of Formula II;

R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

each g is independently 2, 3 or 4;

each h is independently 1, 2, 3 or 4;

m is 0, 1, 2, 3, 4 or 5; if m is more than 1, then L can be the same or different;

m1 is 0, 1, 2 or 3;

k is 0, 1, 2, or 3;

z is 1, 2, or 3;

each R₄ independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

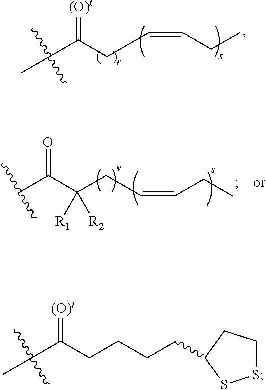

with the proviso that there is at least one

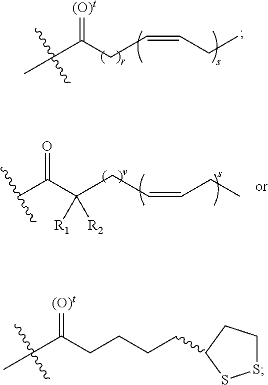

in the compound;

each r is independently 2, 3, or 7;

each s is independently 3, 5, or 6;

each t is independently 0 or 1;

each v is independently 1, 2, or 6;

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

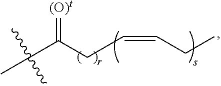

then t must be 0; and
when m, n, o, p, and q are each 0, and W₁ and W₂ are each null, then Z must not be

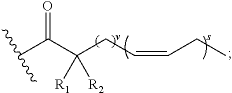

In Formula I and II, any one or more of H may be substituted with a deuterium. It is also understood in Formula I and II that a methyl substituent can be substituted with a C₁-C₆ alkyl.

Also described are pharmaceutical formulations comprising at least one fatty acid non-flushing niacin derivative.

Also described herein are methods of treating a disease susceptible to treatment with a fatty acid non-flushing niacin derivative in a patient in need thereof by administering to the patient an effective amount of a fatty acid non-flushing niacin derivative.

Also described herein are methods of treating metabolic diseases by administering to a patient in need thereof an effective amount of a fatty acid non-flushing niacin derivative.

The invention also includes pharmaceutical compositions that comprise an effective amount of a fatty acid non-flushing niacin derivative and a pharmaceutically acceptable carrier. The compositions are useful for treating or preventing a metabolic disease. The invention includes a fatty acid non-flushing niacin derivative provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION OF THE INVENTION

Metabolic diseases are a wide variety of medical disorders that interfere with a subject's metabolism. Metabolism is the process a subject's body uses to transform food into energy. Metabolism in a subject with a metabolic disease is disrupted in some way. The fatty acid niacin derivatives possess the ability to treat or prevent metabolic diseases.

The fatty acid non-flushing niacin derivatives have been designed to bring together non-flushing niacin analogs and omega-3 fatty acids into a single molecular conjugate. The activity of the fatty acid non-flushing niacin derivatives is substantially greater than the sum of the individual components of the molecular conjugate, suggesting that the activity induced by the fatty acid non-flushing niacin derivatives is synergistic.

### Definitions

The following definitions are used in connection with the fatty acid non-flushing niacin derivatives:

The term “fatty acid non-flushing niacin derivatives” includes any and all possible isomers, stereoisomers, enantiomers, diastereomers, tautomers, pharmaceutically acceptable salts, of the fatty acid niacin derivatives described herein.

The articles “a” and “an” are used in this disclosure to refer to one or more than one

(i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid) and stereoisomers of lipoic acid.

The term “non-flushing niacin” as used herein means the molecule known as non-flushing niacin and any derivative thereof. These non-flushing niacin derivatives are described in WO 2008/016968 and WO 2011/163619. Non-limiting examples of non-flushing niacin derivatives are shown below:

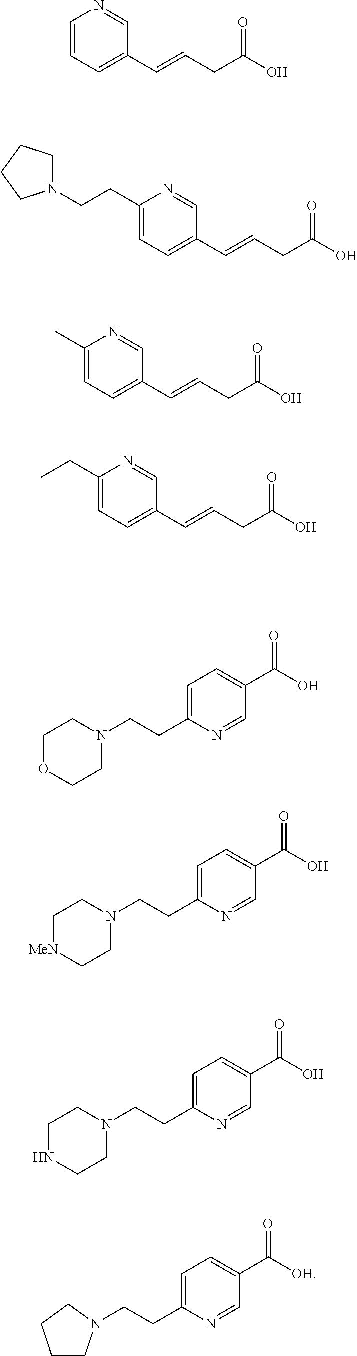

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharmaceutical compositions comprising an effective amount of a fatty acid non-flushing niacin derivative and a pharmaceutically acceptable carrier. The invention includes a fatty acid non-flushing niacin derivative provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a fatty acid non-flushing niacin derivative is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a fatty acid non-flushing niacin derivative.

The following abbreviations are used herein and have the indicated definitions: Boc and BOC are tert-butoxycarbonyl, Boc₂O is di-tert-butyl dicarbonate, BSA is bovine serum albumin, CDI is 1,1′-carbonyldiimidazole, DCC is N,N′-dicyclohexylcarbodiimide, DIEA is N,N-diisopropylethylamine, DMAP is 4-dimethylaminopyridine, DMEM is Dulbecco's Modified Eagle Medium, DMF is N,N-dimethylformamide, DOSS is sodium dioctyl sulfosuccinate, EDC and EDCI are 1-ethyl-3-(3-dimethylaminopropyl)carbodiimide hydrochloride, ELISA is enzyme-linked immunosorbent assay, EtOAc is ethyl acetate, FBS is fetal bovine serum, h is hour, HATU is 2-(7-aza-1H-benzotriazole-1-yl)-1,1,3,3-tetramethyluronium hexafluorophosphate, HIV is human immunodeficiency virus, HPMC is hydroxypropyl methylcellulose, oxone is potassium peroxymonosulfate, Pd/C is palladium on carbon, TFA is trifluoroacetic acid, TGPS is tocopherol propylene glycol succinate, and THF is tetrahydrofuran.

### Compounds

Accordingly in one aspect, the present invention provides a molecular conjugate which comprises a non-flushing niacin and a fatty acid covalently linked, wherein the fatty acid is selected from the group consisting of omega-3 fatty acids and fatty acids that are metabolized in vivo to omega-3 fatty acids, and the conjugate is capable of hydrolysis to produce free non-flushing niacin and free fatty acid.

In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid and tetracosahexaenoic acid. In other embodiments, the fatty acid is selected from eicosapentaenoic acid and docosahexaenoic acid. In some embodiments, the hydrolysis is enzymatic.

In another aspect, the present invention provides compounds according to Formula I and Formula II:

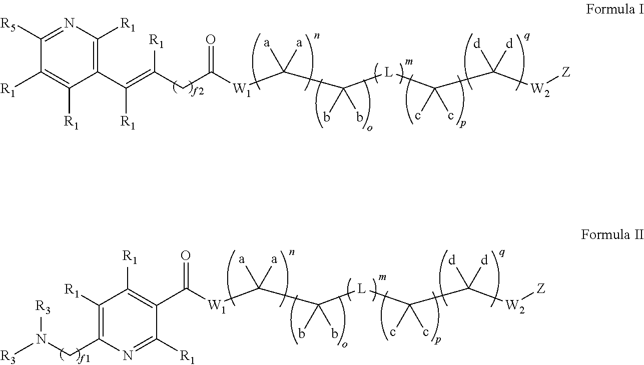

and pharmaceutically acceptable salts, hydrates, solvates, prodrugs, enantiomers, and stereoisomers thereof;

wherein

R₁, R₂, R₃, R₄, R₅, R₆, R, W₁, W₂, L, a, c, b, d, e, f1, f2, g, h, m, m1, n, o, p, q, Z, r, s, t, v and z are as defined above for Formula I and Formula II,

with the proviso that there is at least one of

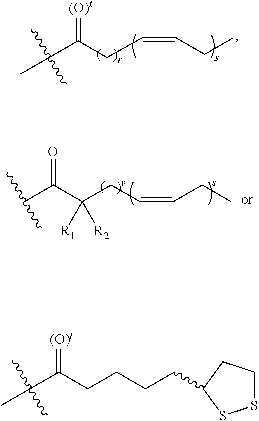

in the compound.

In some embodiments, one Z is

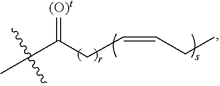

and r is 2.

In some embodiments, one Z is

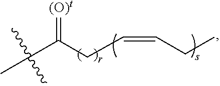

and r is 3.

In some embodiments, one Z is

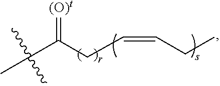

and r is 7.

In other embodiments, one Z is

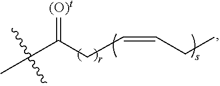

and s is 3.

In some embodiments, one Z is

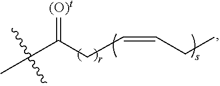

and s is 5.

In some embodiments, one Z is

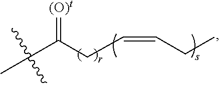

and s is 6.

In some embodiments, one Z is

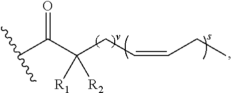

and v is 1.

In other embodiments, one Z is

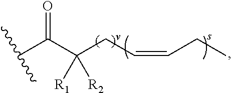

and v is 2.

In some embodiments, one Z is

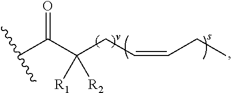

and v is 6.

In some embodiments, one Z is

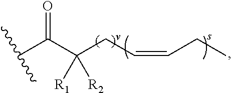

and s is 3.

In some embodiments, one Z is

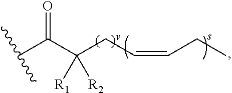

and s is 5.

In other embodiments, one Z is

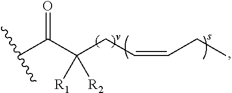

and s is 6.

In other embodiments, Z is

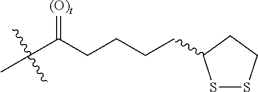

and t is 1.

In some embodiments, Z is

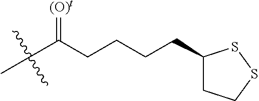

and t is 1.

In some embodiments, W₁ is NH.

In some embodiments, W₂ is NH.

In some embodiments, W₁ is O.

In some embodiments, W₂ is O.

In some embodiments, W₁ is null.

In some embodiments, W₂ is null.

In some embodiments, W₁ and W₂ are each NH.

In some embodiments, W₁ and W₂ are each null.

In some embodiments, W₁ is O and W₂ is NH.

In some embodiments, W₁ and W₂ are each NR, and R is CH₃.

In some embodiments, m is 0.

In other embodiments, m is 1.

In other embodiments, m is 2.

In some embodiments, L is —S— or —S—S—.

In some embodiments, L is —O—.

In some embodiments, L is —C(O)—.

In some embodiments, L is heteroaryl.

In some embodiments, L is heterocycle.

In some embodiments, L is

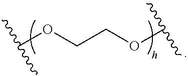

In some embodiments, L is

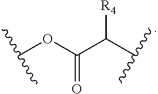

In some embodiments, L is

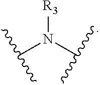

In some embodiments, L is

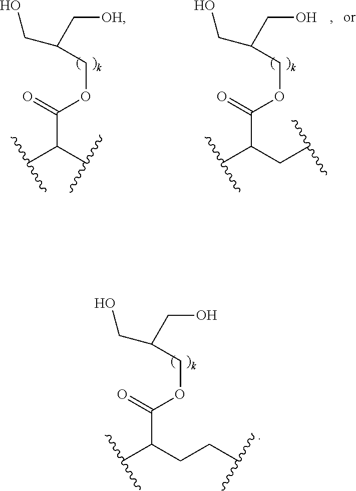

In some embodiments, L is

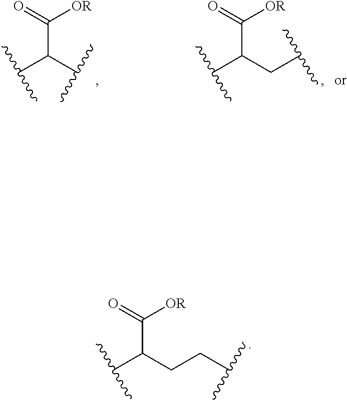

In some embodiments, L is

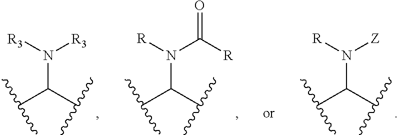

In some embodiments, L is

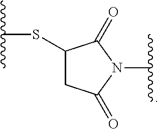

In some embodiments, L is

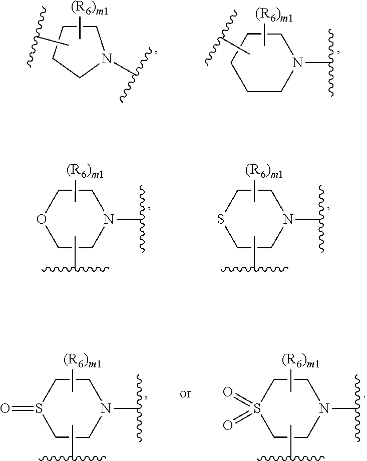

In some embodiments, L is

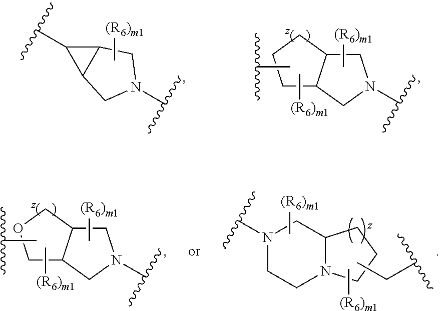

In some embodiments, L is

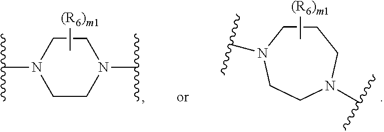

In some embodiments, L is

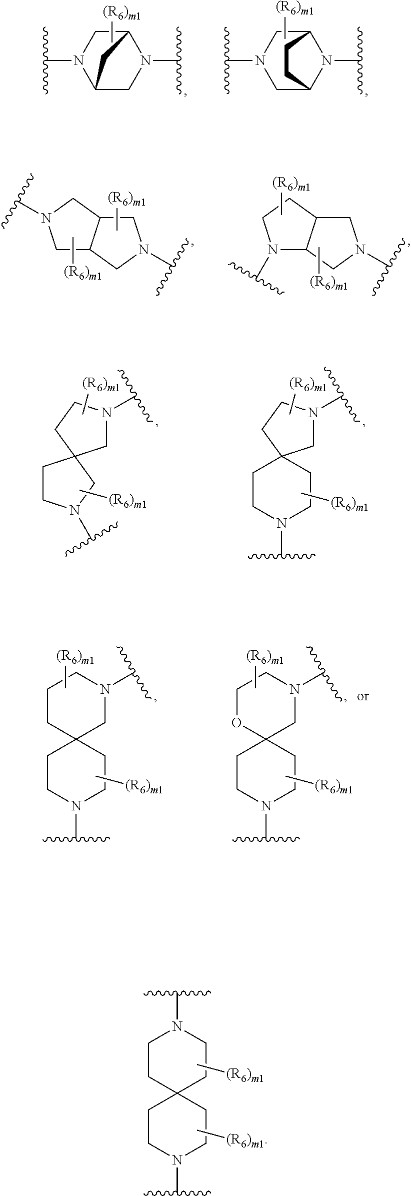

In other embodiments, one of n, o, p, and q is 1.

In some embodiments, two of n, o, p, and q are each 1.

In other embodiments, three of n, o, p, and q are each 1.

In some embodiments n, o, p, and q are each 1.

In some embodiments, one d is C(O)OR.

In some embodiments, r is 2 and s is 6.

In some embodiments, r is 3 and s is 5.

In some embodiments, t is 1.

In some embodiments, W₁ and W₂ are each NH, m is 0, n and o are each 1, and p and q are each 0.

In some embodiments, W₁ and W₂ are each NH, m is 1, n, o, p, and q are each 1, and L is O.

In some embodiments, W₁ and W₂ are each NH, m is 1, n, o, p, and q are each 1, and

L is

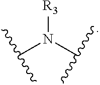

In some embodiments, W₁ and W₂ are each NH, m is 1, n, o, p, and q are each 1, and L is —S—S—.

In some embodiments, W₁ and W₂ are each NH, m is 1, n and o are each 0, p and q are each 1, and L is

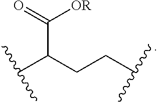

In some embodiments, W₁ and W₂ are each NH, m is 1, k is O, n and o are each 0, p and q are each 1, and L is

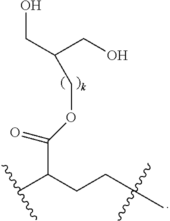

In some embodiments, W₁ and W₂ are each NH, m is 1, n and o are each 1, p and q are each 0, and L is

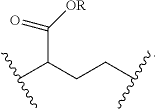

In some embodiments, W₁ and W₂ are each NH, m is 1, k is 0, n is 1, o, p and q are each 0, and L is

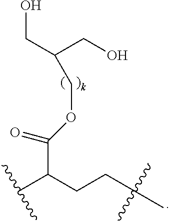

In some embodiments, W₁ and W₂ are each NH, m is 1, n, o, and p are each 0, and q is 1, and L is

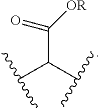

In some embodiments, W₁ and W₂ are each NH, m is 1, k is 1, n, o, and p are each 0, and q is 1, and L is

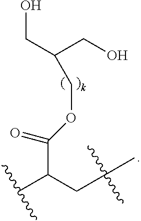

In some embodiments, W₁ and W₂ are each NH, m is 1, n is 1, and o, p, and q are each 0, and L is

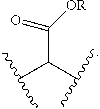

In some embodiments, W₁ and W₂ are each NH, m is 1, k is 1, o, p, and q are each 0, and L is

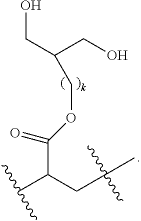

In some embodiments, W₁ and W₂ are each NH, m is 1, n, o, p, and q are each 1, and L is

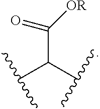

In some embodiments, W₁ and W₂ are each NH, m is 1, n, o, p, and q are each 1, and L is

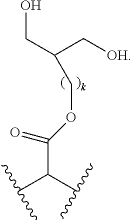

In some embodiments, W₁ and W₂ are each NH, m is 0, k is 1, o and p are each 1, and

q is O.

In some embodiments, W₁ and W₂ are each NH, m is 0, n, o, p, and q are each 1.

In some embodiments, W₁ and W₂ are each NH, m is 0, n and o are each 1, p and q are each 0, and each a is CH₃.

In some embodiments, W₁ and W₂ are each NH, m is 0, n and o are each 1, p and q are each 0, and each b is CH₃.

In some embodiments, W₁ and W₂ are each NH, m is 1, n, o, p, and q are each 1, R₃ is H, and L is

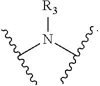

In some embodiments, W₁ and W₂ are each NH, m is 1, n, p and q are each 1, and o is 2, R₃ is H, and L is

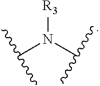

In some embodiments, W₁ and W₂ are each NH, m is 1, n, o, p are each 1, and q is 2, and L is

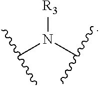

In some embodiments, W₁ and W₂ are each NH, m is 1, n, o, p, and q are each 1, and L is

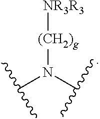

In some embodiments, W₁ and W₂ are each NH, m is 1, n and p are each 1, and o and q are each 0, and L is —C(O)—.

In some embodiments, W₁ and W₂ are each NH, m is 1, n and p are each 1, and o, and q are each 0, and L is

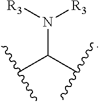

In some embodiments, W₁ and W₂ are each NH, m is 1, n, o, p, q are each 1, and L is

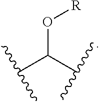

In some embodiments, W₁ and W₂ are each NH, m is 1, n, o, p, and q are each 1, h is 1, and L is

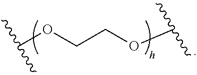

In some embodiments, W₁ and W₂ are each NH, m is 1, n, o, p, and q are each 1, and L is —S—.

In some embodiments, W₁ and W₂ are each NH, m is 1, n, o, p are each 0, q is 1, one d is —CH₃, and L is

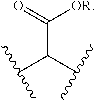

In some embodiments, W₁ and W₂ are each NH, m is 2, n, o, p, and q are each 0, one L is

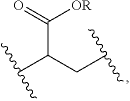

and
one L is

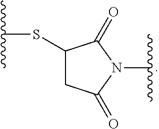

In some embodiments, m is 0, n, o, p, and q are each 0, and W₁ and W₂ are taken together to form an optionally substituted piperazine group.

In some embodiments, m is 1, n, o, p, and q are each 0, W₁ and W₂ are each null, and L is

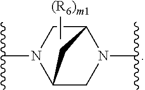

In some embodiments, m is 1, n and p are each 1, o and q are each 0, W₁ and W₂ are each NH, and L is C₃-C₆ cycloalkyl.

In some embodiments, m is 1, n is 1, o, p, and q are each 0, W₁ and W₂ are each NH, and L is C₃-C₆ cycloalkyl.

In some embodiments, m is 1, n, o, p, are each 0, q is 1, W₁ and W₂ are each NH, and L is C₃-C₆ cycloalkyl.

In some embodiments, m is 1, n, o, p, and q are each 0, W₁ is NH, W₂ is null, and L is

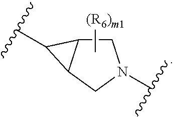

In some embodiments, m is 1, n o, p, and q are each 0, W₁ is null, W₂ is NH, and L is

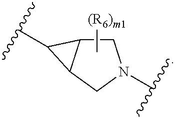

In some embodiments, m is 1, n o, p, and q are each 0, W₁ is NH, W₂ is null, and L is

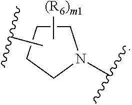

In some embodiments, m is 1, n o, p, and q are each 0, W₁ is null, W₂ is NH, and L is

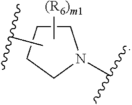

In some embodiments, m is 1, n is 1, o, p, and q are each 0, W₁ is NH, W₂ is null, and L is

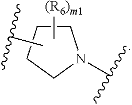

In some embodiments, m is 1, n, o, p, are each 0, q is 1, W₁ is null, W₂ is NH, and L is

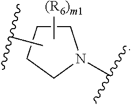

In some embodiments, m is 1, n, o, p, and q are each 0, W₁ is NH, W₂ is null, and L is

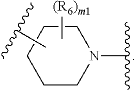

In some embodiments, m is 1, n, o, p, and q are each 0, W₁ is null, W₂ is NH, and L is

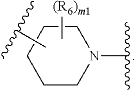

In some embodiments, m is 1, n is 1, o, p, and q are each 0, W₁ is NH, W₂ is null, and L is

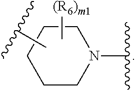

In some embodiments, m is 1, n, o, p, are each 0, q is 1, W₁ is null, W₂ is NH, and L is

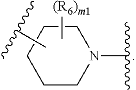

In some embodiments, m is 1, n is 1, o, p, and q are each 0, W₁ is NH, W₂ is null, and L is

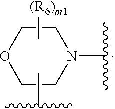

In some embodiments, m is 1, n, o, p, are each 0, q is 1, W₁ is null, W₂ is NH, and L is

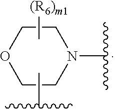

In some embodiments, m is 1, n, o, p, q are each 0, W₁ and W₂ is null, and L is

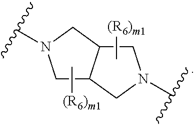

In some embodiments, m is 1, n, o, p, q are each 0, W₁ and W₂ is null, and L is

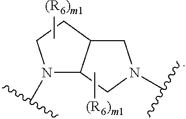

In some embodiments, m is 1, n, o, p, q are each 0, W₁ is NH, W₂ is null, and L is

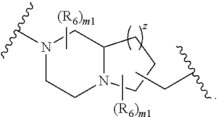

In some embodiments, m is 1, n, o, p, q are each 0, W₁ is null, W₂ is NH, and L is

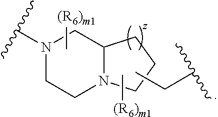

In some embodiments, m is 1, n, o, p, are each 0, q is 1, W₁ and W₂ are each and NH, is null, L is

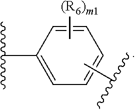

In some embodiments, m is 1, n, o, p, are each 0, q is 1, W₁ and W₂ are each NH, is null, and L is a heteroaryl.

In some embodiments, when W₁ and W₂ are each NH, then f2=1.

In some embodiments, when f2=1, then R₅═H.

In some embodiments, when f2=1, then R₅=Me.

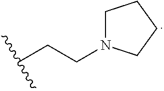

In some embodiments, when f2=1, then R₅=

In some embodiments, when f1=2 then the R₃ groups in (R₃)₂N— can be taken together to form a morpholine.

In some embodiments, when f1=2 then the R3 groups in (R3)₂N— can be taken together to form a pyrrolidine.

In some of the foregoing embodiments, r is 2, s is 6 and t is 1.

In some of the foregoing embodiments, r is 3, s is 5 and t is 1.

In some of the foregoing embodiments, Z is

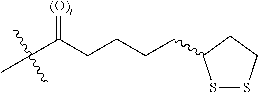

and

t is 1.

In Formula I and Formula II, any one or more of H may be substituted for a deuterium. It is also understood in Formula I and Formula II that a methyl substituent may have as a substituent a C₁-C₆ alkyl.

In other illustrative embodiments, compounds of Formula I are as set forth below:
(4Z,7Z,10Z,13Z,16Z,19Z)-N-(2-((E)-4-(pyridin-3-yl)but-3-enamido)ethyl)docosa-4,7,10,13,16,19-hexaenamide (I-1);
(5Z,8Z,11Z,14Z,17Z)-N-(2-((E)-4-(pyridin-3-yl)but-3-enamido)ethyl)icosa-5,8,11,14,17-pentaenamide (I-2);
(5Z,8Z,11Z,14Z,17Z)-N-(2-(2-((E)-4-(pyridin-3-yl)but-3-enamido)ethoxy)ethyl)icosa-5,8,11,14,17-pentaenamide (I-3);
(5Z,8Z,11Z,14Z,17Z)-N-(2-(2-((E)-4-(pyridin-3-yl)but-3-enamido)ethylamino)ethyl)icosa-5,8,11,14,17-pentaenamide (I-4);
(5Z,8Z,11Z,14Z,17Z)-N-(2-(methyl(2-((E)-4-(pyridin-3-yl)but-3-enamido)ethyl)amino)ethyl)icosa-5,8,11,14,17-pentaenamide (I-5);
(5Z,8Z,11Z,14Z,17Z)-N-(2-(2-(2-((E)-4-(pyridin-3-yl)but-3-enamido)ethyl)disulfanyl)ethyl)icosa-5,8,11,14,17-pentaenamide (I-6);
(S)-6-((5Z,8Z,11Z,14Z,17Z)-icosa-5,8,11,14,17-pentaenamido)-2-((E)-4-(pyridin-3-yl)but-3-enamido)hexanoic acid (I-7);
(S)-2-((5Z,8Z,11Z,14Z,17Z)-icosa-5,8,11,14,17-pentaenamido)-6-((E)-4-(pyridin-3-yl)but-3-enamido)hexanoic acid (I-8);
(S)-1,3-dihydroxypropan-2-yl 6-((5Z,8Z,11Z,14Z,17Z)-icosa-5,8,11,14,17-pentaenamido)-2-((E)-4-(pyridin-3-yl)but-3-enamido)hexanoate (I-9);
(S)-1,3-dihydroxypropan-2-yl 2-((5Z,8Z,11Z,14Z,17Z)-icosa-5,8,11,14,17-pentaenamido)-6-((E)-4-(pyridin-3-yl)but-3-enamido)hexanoate (I-10);
(S)-5-((4Z,7Z,10Z,13Z,16Z,19Z)-docosa-4,7,10,13,16,19-hexaenamido)-2-((E)-4-(pyridin-3-yl)but-3-enamido)pentanoic acid (I-11);
(S)-1,3-dihydroxypropan-2-yl 5-((4Z,7Z,10Z,13Z,16Z,19Z)-docosa-4,7,10,13,16,19-hexaenamido)-2-((E)-4-(pyridin-3-yl)but-3-enamido)pentanoate (I-12);
(4Z,7Z,10Z,13Z,16Z,19Z)-N-(2-((E)-4-(6-methylpyridin-3-yl)but-3-enamido)ethyl)docosa-4,7,10,13,16,19-hexaenamide (I-13);
(5Z,8Z,11Z,14Z,17Z)-N-(2-((E)-4-(6-methylpyridin-3-yl)but-3-enamido)ethyl)icosa-5,8,11,14,17-pentaenamide (I-14);
(5Z,8Z,11Z,14Z,17Z)-N-(2-(2-((E)-4-(6-methylpyridin-3-yl)but-3-enamido)ethoxy)ethyl)icosa-5,8,11,14,17-pentaenamide (I-15);
(5Z,8Z,11Z,14Z,17Z)-N-(2-(2-((E)-4-(6-methylpyridin-3-yl)but-3-enamido)ethylamino)ethyl)icosa-5,8,11,14,17-pentaenamide (I-16);
(5Z,8Z,11Z,14Z,17Z)-N-(2-(methyl(2-((E)-4-(6-methylpyridin-3-yl)but-3-enamido)ethyl)amino)ethyl)icosa-5,8,11,14,17-pentaenamide (I-17);
(5Z,8Z,11Z,14Z,17Z)-N-(2-(2-(2-((E)-4-(6-methylpyridin-3-yl)but-3-enamido)ethyl)disulfanyl)ethyl)icosa-5,8,11,14,17-pentaenamide (I-18);
(S)-6-((5Z,8Z,11Z,14Z,17Z)-icosa-5,8,11,14,17-pentaenamido)-2-((E)-4-(6-methylpyridin-3-yl)but-3-enamido)hexanoic acid (I-19);
(S)-2-((5Z,8Z,11Z,14Z,17Z)-icosa-5,8,11,14,17-pentaenamido)-6-((E)-4-(6-methylpyridin-3-yl)but-3-enamido)hexanoic acid (I-20);
(S)-1,3-dihydroxypropan-2-yl 6-((5Z,8Z,11Z,14Z,17Z)-icosa-5,8,11,14,17-pentaenamido)-2-((E)-4-(6-methylpyridin-3-yl)but-3-enamido)hexanoate (I-21);
(S)-1,3-dihydroxypropan-2-yl 2-((5Z,8Z,11Z,14Z,17Z)-icosa-5,8,11,14,17-pentaenamido)-6-((E)-4-(6-methylpyridin-3-yl)but-3-enamido)hexanoate (I-22);
(S)-5-((4Z,7Z,10Z,13Z,16Z,19Z)-docosa-4,7,10,13,16,19-hexaenamido)-2-((E)-4-(6-methylpyridin-3-yl)but-3-enamido)pentanoic acid (I-23);
(S)-1,3-dihydroxypropan-2-yl 5-((4Z,7Z,10Z,13Z,16Z,19Z)-docosa-4,7,10,13,16,19-hexaenamido)-2-((E)-4-(6-methylpyridin-3-yl)but-3-enamido)pentanoate (I-24);
(4Z,7Z,10Z,13Z,16Z,19Z)-N-(2-((E)-4-(6-(2-(pyrrolidin-1-yl)ethyl)pyridin-3-yl)but-3-enamido)ethyl)docosa-4,7,10,13,16,19-hexaenamide (I-25);
(5Z,8Z,11Z,14Z,17Z)-N-(2-((E)-4-(6-(2-(pyrrolidin-1-yl)ethyl)pyridin-3-yl)but-3-enamido)ethyl)icosa-5,8,11,14,17-pentaenamide (I-26);
(5Z,8Z,11Z,14Z,17Z)-N-(2-(2-((E)-4-(6-(2-(pyrrolidin-1-yl)ethyl)pyridin-3-yl)but-3-enamido)ethoxy)ethyl)icosa-5,8,11,14,17-pentaenamide (I-27);
(5Z,8Z,11Z,14Z,17Z)-N-(2-(2-((E)-4-(6-(2-(pyrrolidin-1-yl)ethyl)pyridin-3-yl)but-3-enamido)ethylamino)ethyl)icosa-5,8,11,14,17-pentaenamide (I-28);
(5Z,8Z,11Z,14Z,17Z)-N-(2-(methyl(2-((E)-4-(6-(2-(pyrrolidin-1-yl)ethyl)pyridin-3-yl)but-3-enamido)ethyl)amino)ethyl)icosa-5,8,11,14,17-pentaenamide (I-29);
(5Z,8Z,11Z,14Z,17Z)-N-(2-(2-(2-((E)-4-(6-(2-(pyrrolidin-1-yl)ethyl)pyridin-3-yl)but-3-enamido)ethyl)disulfanyl)ethyl)icosa-5,8,11,14,17-pentaenamide (I-30);
(S)-6-((5Z,8Z,11Z,14Z,17Z)-icosa-5,8,11,14,17-pentaenamido)-2-((E)-4-(6-(2-(pyrrolidin-1-yl)ethyl)pyridin-3-yl)but-3-enamido)hexanoic acid (I-31);
(S)-2-((5Z,8Z,11Z,14Z,17Z)-icosa-5,8,11,14,17-pentaenamido)-6-((E)-4-(6-(2-(pyrrolidin-1-yl)ethyl)pyridin-3-yl)but-3-enamido)hexanoic acid (I-32);
(S)-1,3-dihydroxypropan-2-yl 6-((5Z,8Z,11Z,14Z,17Z)-icosa-5,8,11,14,17-pentaenamido)-2-((E)-4-(6-(2-(pyrrolidin-1-yl)ethyl)pyridin-3-yl)but-3-enamido)hexanoate (I-33);
(S)-1,3-dihydroxypropan-2-yl 2-((5Z,8Z,11Z,14Z,17Z)-icosa-5,8,11,14,17-pentaenamido)-6-((E)-4-(6-(2-(pyrrolidin-1-yl)ethyl)pyridin-3-yl)but-3-enamido)hexanoate (I-34);
(S)-5-((4Z,7Z,10Z,13Z,16Z,19Z)-docosa-4,7,10,13,16,19-hexaenamido)-2-((E)-4-(6-(2-(pyrrolidin-1-yl)ethyl)pyridin-3-yl)but-3-enamido)pentanoic acid (I-35);
(S)-1,3-dihydroxypropan-2-yl 5-((4Z,7Z,10Z,13Z,16Z,19Z)-docosa-4,7,10,13,16,19-hexaenamido)-2-((E)-4-(6-(2-(pyrrolidin-1-yl)ethyl)pyridin-3-yl)but-3-enamido)pentanoate (I-36);
(R,E)-5-(1,2-dithiolan-3-yl)-N-(2-(4-(pyridin-3-yl)but-3-enamido)ethyl)pentanamide (I-37);
(R,E)-5-(1,2-dithiolan-3-yl)-N-(2-(4-(6-methylpyridin-3-yl)but-3-enamido)ethyl)pentanamide (I-38); and
(R,E)-5-(1,2-dithiolan-3-yl)-N-(2-(4-(6-(2-(pyrrolidin-1-yl)ethyl)pyridin-3-yl)but-3-enamido)ethyl)pentanamide (I-39).

In other illustrative embodiments, compounds of Formula II are as set forth below:
N-(2-((4Z,7Z,10Z,13Z,16Z,19Z)-docosa-4,7,10,13,16,19-hexaenamido)ethyl)-6-(2-morpholinoethyl)nicotinamide (I-40);
N-(2-((5Z,8Z,11Z,14Z,17Z)-icosa-5,8,11,14,17-pentaenamido)ethyl)-6-(2-morpholinoethyl)nicotinamide (I-41);
(R)-N-(2-(5-(1,2-dithiolan-3-yl)pentanamido)ethyl)-6-(2-morpholinoethyl)nicotinamide (I-42);
N-(2-((2-((4Z,7Z,10Z,13Z,16Z,19Z)-docosa-4,7,10,13,16,19-hexaenamido)ethyl)amino)ethyl)-6-(2-morpholinoethyl)nicotinamide (I-43);
N-(2-((2-((5Z,8Z,11Z,14Z,17Z)-icosa-5,8,11,14,17-pentaenamido)ethyl)amino)ethyl)-6-(2-morpholinoethyl)nicotinamide (I-44);
N-(2-((2-((5Z,8Z,11Z,14Z,17Z)-icosa-5,8,11,14,17-pentaenamido)ethyl)(methyl)amino)ethyl)-6-(2-morpholinoethyl)nicotinamide (I-45);
N-(2-(2-((5Z,8Z,11Z,14Z,17Z)-icosa-5,8,11,14,17-pentaenamido)ethoxy)ethyl)-6-(2-morpholinoethyl)nicotinamide (I-46);
(S)-6-((5Z,8Z,11Z,14Z,17Z)-icosa-5,8,11,14,17-pentaenamido)-2-(6-(2-morpholinoethyl)nicotinamido)hexanoic acid (I-47);
(S)-2-((5Z,8Z,11Z,14Z,17Z)-icosa-5,8,11,14,17-pentaenamido)-6-(6-(2-morpholinoethyl)nicotinamido)hexanoic acid (I-48);
(S)-1,3-dihydroxypropan-2-yl 2-((5Z,8Z,11Z,14Z,17Z)-icosa-5,8,11,14,17-pentaenamido)-6-(6-(2-morpholinoethyl)nicotinamido)hexanoate (I-49);
(S)-1,3-dihydroxypropan-2-yl 6-((5Z,8Z,11Z,14Z,17Z)-icosa-5,8,11,14,17-pentaenamido)-2-(6-(2-morpholinoethyl)nicotinamido)hexanoate (I-50);
N-(2-((5Z,8Z,11Z,14Z,17Z)-icosa-5,8,11,14,17-pentaenamido)ethyl)-6-(2-(pyrrolidin-1-yl)ethyl)nicotinamide (I-51);
(R)-N-(2-(5-(1,2-dithiolan-3-yl)pentanamido)ethyl)-6-(2-(pyrrolidin-1-yl)ethyl)nicotinamide (I-52);
N-(2-((2-((5Z,8Z,11Z,14Z,17Z)-icosa-5,8,11,14,17-pentaenamido)ethyl)amino)ethyl)-6-(2-(pyrrolidin-1-yl)ethyl)nicotinamide (I-53);
N-(2-((2-((5Z,8Z,11Z,14Z,17Z)-icosa-5,8,11,14,17-pentaenamido)ethyl)(methyl)amino)ethyl)-6-(2-(pyrrolidin-1-yl)ethyl)nicotinamide (I-54);
N-(2-(2-((5Z,8Z,11Z,14Z,17Z)-icosa-5,8,11,14,17-pentaenamido)ethoxy)ethyl)-6-(2-(pyrrolidin-1-yl)ethyl)nicotinamide (I-55);
(S)-6-((5Z,8Z,11Z,14Z,17Z)-icosa-5,8,11,14,17-pentaenamido)-2-(6-(2-(pyrrolidin-1-yl)ethyl)nicotinamido)hexanoic acid (I-56);
(S)-2-((5Z,8Z,11Z,14Z,17Z)-icosa-5,8,11,14,17-pentaenamido)-6-(6-(2-(pyrrolidin-1-yl)ethyl)nicotinamido)hexanoic acid (I-57);
(S)-1,3-dihydroxypropan-2-yl 6-((5Z,8Z,11Z,14Z,17Z)-icosa-5,8,11,14,17-pentaenamido)-2-(6-(2-(pyrrolidin-1-yl)ethyl)nicotinamido)hexanoate (I-58); and
(S)-1,3-dihydroxypropan-2-yl 2-((5Z,8Z,11Z,14Z,17Z)-icosa-5,8,11,14,17-pentaenamido)-6-(6-(2-(pyrrolidin-1-yl)ethyl)nicotinamido)hexanoate (I-59).

### Methods for Using Fatty Acid Non-Flushing Niacin Derivatives

The invention also includes methods for treating metabolic diseases such as the treatment or prevention of metabolic diseases including atherosclerosis, dyslipidemia, coronary heart disease, hypercholesterolemia, Type 2 diabetes, elevated cholesterol, metabolic syndrome and cardiovascular disease.

In one embodiment, the method comprises contacting a cell with a fatty acid non-flushing niacin derivative in an amount sufficient to decrease the release of triglycerides or VLDL or LDL or cause an increase in reverse cholesterol transport or increase HDL concentrations.

Also provided in the invention is a method for inhibiting, preventing, or treating a metabolic disease, or symptoms of a metabolic disease, in a subject. Examples of such disorders include, but are not limited to atherosclerosis, dyslipidemia, hypertriglyceridemia, hypertension, heart failure, cardiac arrhythmias, low HDL levels, high LDL levels, sudden death, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, carotid atherosclerosis, peripheral arterial disease, diabetic nephropathy, hypercholesterolemia in HIV infection, acute coronary syndrome (ACS), non-alcoholic fatty liver disease, arterial occlusive diseases, cerebral arteriosclerosis, cerebrovascular disorders, myocardial ischemia, and diabetic autonomic neuropathy.

In some embodiments, the subject is administered an effective amount of a fatty acid non-flushing niacin derivative.

The invention also includes pharmaceutical compositions useful for treating or preventing a metabolic disease, or for inhibiting a metabolic disease, or more than one of these activities. The compositions can be suitable for internal use and comprise an effective amount of a fatty acid non-flushing niacin derivative and a pharmaceutically acceptable carrier. The fatty acid non-flushing niacin derivatives are especially useful in that they demonstrate very low peripheral toxicity or no peripheral toxicity.

The fatty acid non-flushing niacin derivatives can each be administered in amounts that are sufficient to treat or prevent a metabolic disease or prevent the development thereof in subjects.

Administration of the fatty acid non-flushing niacin derivatives can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid non-flushing niacin derivative and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid non-flushing niacin derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid non-flushing niacin derivatives.

The fatty acid non-flushing niacin derivatives can be also formulated as a suppository that can be prepared from fatty emulsions or suspensions; using polyalkylene glycols such as propylene glycol, as the carrier.

The fatty acid non-flushing niacin derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are herein incorporated by reference in their entirety.

Fatty acid non-flushing niacin derivatives can also be delivered by the use of monoclonal antibodies as individual carriers to which the fatty acid niacin derivatives are coupled. The fatty acid non-flushing niacin derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the fatty acid niacin derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid non-flushing niacin derivatives are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90% of the fatty acid non-flushing niacin derivative by weight or volume.

The dosage regimen utilizing the fatty acid non-flushing niacin derivative is selected in accordance with a variety of factors including type, species, age, weight, sex and medical condition of the patient; the severity of the condition to be treated; the route of administration; the renal or hepatic function of the patient; and the particular fatty acid niacin derivative employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the fatty acid non-flushing niacin derivative per day. Compositions for in vivo or in vitro use can contain about 20, 50, 75, 100, 150, 250, 500, 750, 1,000, 1,250, 2,500, 3,500, or 5,000 mg of the fatty acid non-flushing niacin derivative. In one embodiment, the compositions are in the form of a tablet that can be scored. Effective plasma levels of the fatty acid non-flushing niacin derivative can range from about 5 ng/mL to about 5000 ng/mL. Appropriate dosages of the fatty acid niacin derivatives can be determined as set forth in Goodman, L. S.; Gilman, A. The Pharmacological Basis of Therapeutics, 5th ed.; MacMillan: New York, 1975, pp. 201-226.

Fatty acid non-flushing niacin derivatives can be administered in a single daily dose, or the total daily dosage can be administered in divided doses of two, three or four times daily. Furthermore, fatty acid non-flushing niacin derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid non-flushing niacin derivative ranges from about 0.1% to about 15%, w/w or w/v.

### Methods of Making

### Methods for Making the Fatty Acid Non Flushing Niacin Derivatives

Examples of synthetic pathways useful for making fatty acid non-flushing niacin derivatives of Formula I and Formula II are set forth in the Examples below and generalized in Schemes 1-10.

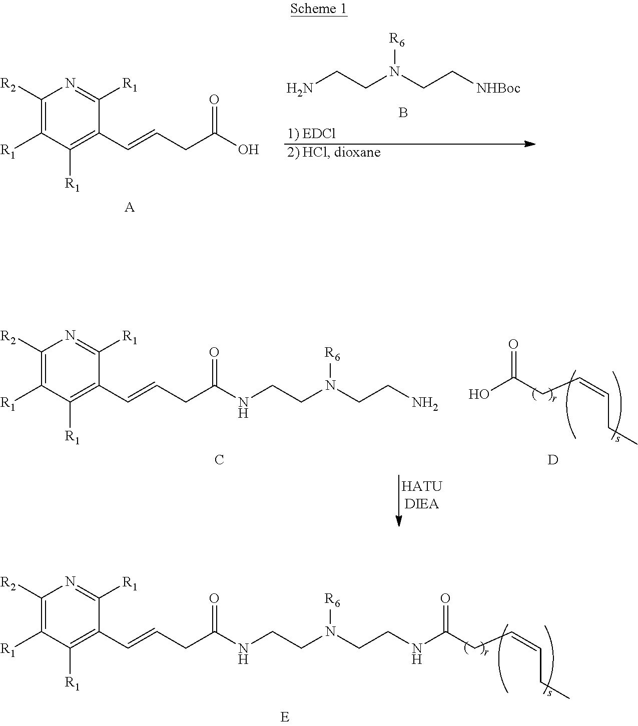

wherein R₁, R₂, R₃, R₆, r, and s are as defined above.

The mono-BOC protected amine of the formula B can be obtained from commercial sources or prepared according to the procedures outlined in Krapcho et al. Synthetic Communications 1990, 20, 2559-2564. Compound A can be amidated with the amine B using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound C. Activation of compound C with a coupling agent such as HATU in the presence of an amine such as DIEA followed by addition of a fatty acid of formula D affords compounds of the formula E. To those familiar in the art, the fatty acid D used in this scheme, as well as the remaining schemes, can also be lipoic acid. Also, to those familiar in the art, compound A in Scheme 1, as well as in subsequent schemes, can also be substituted with compound A1 shown below (wherein R₁, R₃ and f1 are as defined above):

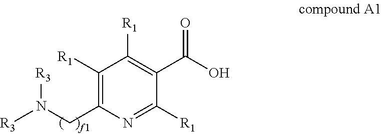

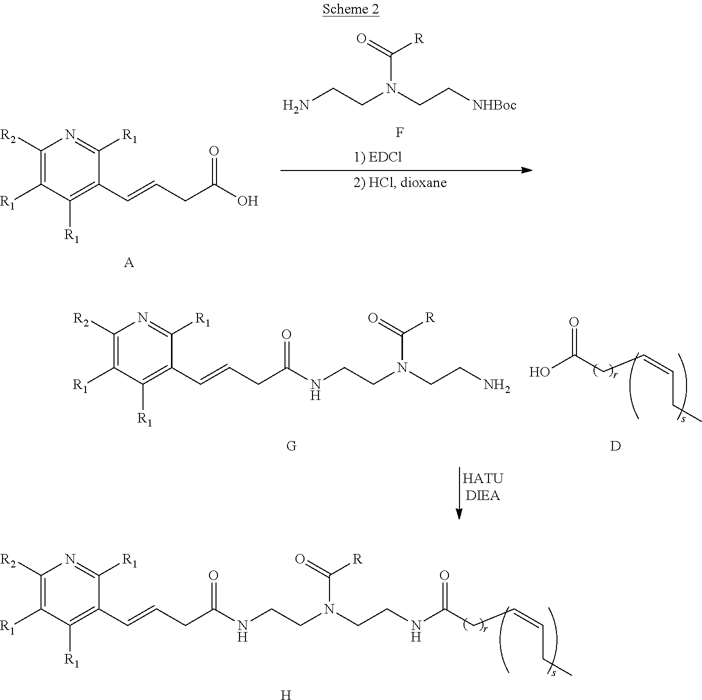

wherein R, R₁, R₂, r, and s are as defined above.

The acylated amine of the formula F can be prepared using the procedures outlined in Andruszkiewicz et al. Synthetic Communications 2008, 38, 905-913. Compound A can be amidated with the amine F using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound G. Activation of compound G with a coupling agent such as HATU in the presence of an amine such as DIEA followed by addition of a fatty acid of formula D affords compounds of the formula H.

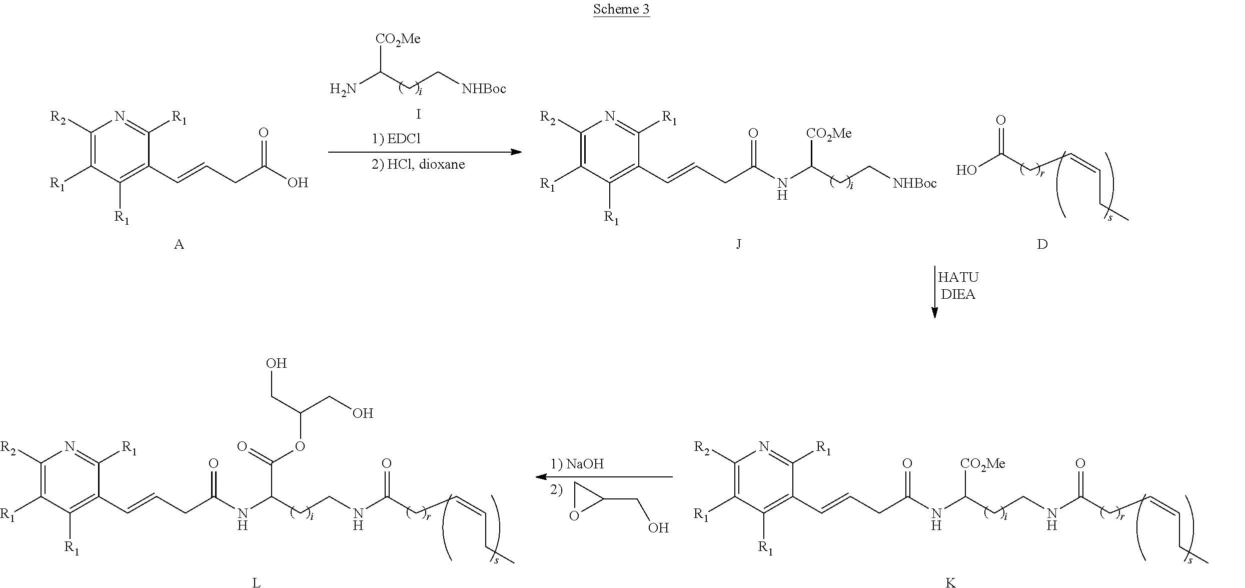

wherein R₁, R₂, r and s are as defined above.

Compound A can be amidated with the corresponding amine I (where i=0, 1, 2 or 3) using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound J. Activation of compound J with a coupling agent such as HATU in the presence of an amine such as DIEA followed by addition of a fatty acid of formula D affords compounds of the formula K. Hydrolysis of the ester under basic conditions such as NaOH or LiOH produces the corresponding acid, which can be coupled with glycidol to afford compounds of the formula L.

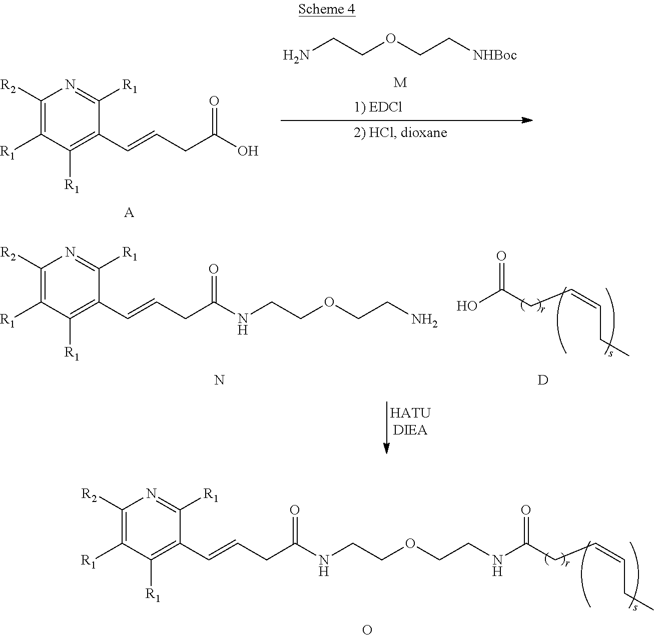

wherein R₁, R₂, r and s are as defined above.

The amine M can be prepared according to the procedures outlined in Dahan et al. J. Org. Chem. 2007, 72, 2289-2296. Compound A can be coupled with the amine M using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound N. Activation of compound N with a coupling agent such as HATU in the presence of an amine such as DIEA followed by addition of a fatty acid of formula D affords compounds of the formula O.

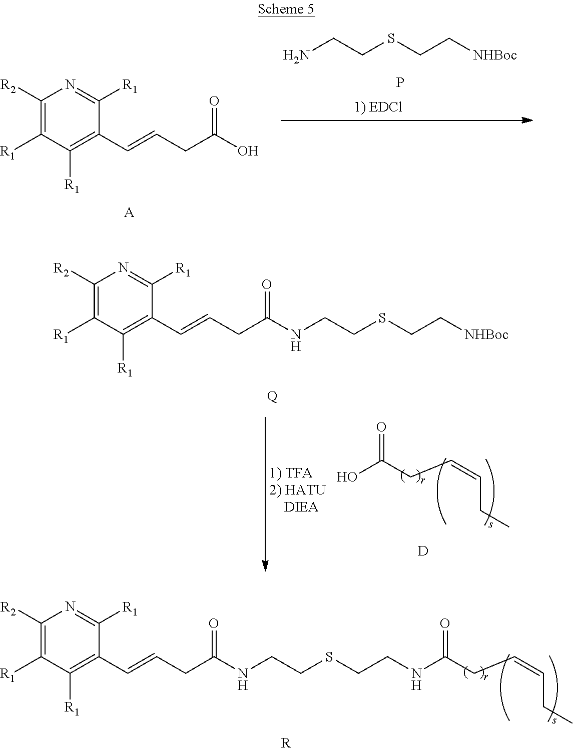

wherein R₁, R₂, r and s are as defined above.

Compound A can be amidated with the commercially available amine P using a coupling reagent such as DCC, CDI, EDC, or optionally with a tertiary amine base and/or catalyst, e.g., DMAP, to afford compound Q. The BOC group in compound Q can be removed with acids such as TFA or HCl in a solvent such as CH₂Cl₂ or dioxane and the resulting amine can be coupled with a fatty acid of formula D using a coupling agent such as HATU in the presence of an amine such as DIEA to afford compounds of the formula R. To those skilled in the art, the sulfur group in formula Q can be oxidized to the corresponding sulfoxide or sulfone using an oxidizing agent such as H₂O₂ or oxone.

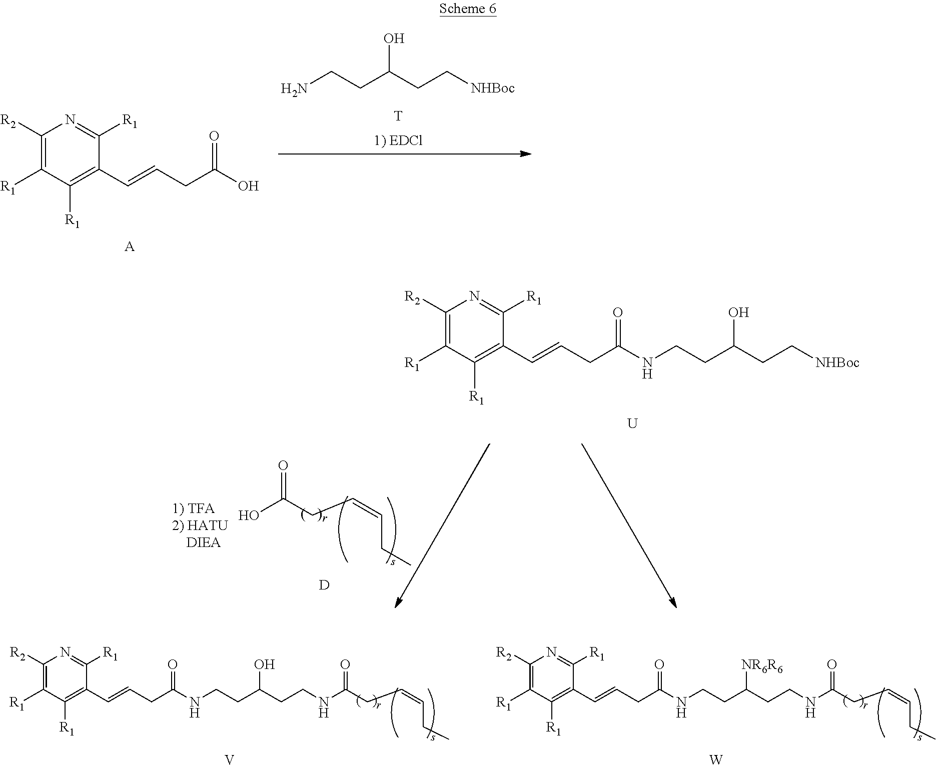

wherein R₁, R₂, R₆, r, and s are as defined above.

The amine T can be prepared from the commercially available diamine according to the procedures outlined in Dahan et al. J. Org. Chem. 2007, 72, 2289-2296. Compound A can be amidated with the amine T using a coupling reagent such as DCC, CDI, EDC, or optionally with a tertiary amine base and/or catalyst, e.g., DMAP, to afford compound U. The BOC group of compound U can be removed with acids such as TFA or HCl in a solvent such as CH₂Cl₂ or dioxane and the resulting amine can be coupled with a fatty acid of formula D using HATU in the presence of an amine such as DIEA to afford compounds of the formula V. To those skilled in the art, the hydroxyl group in compound U can be further acylated or converted to an amino group by standard mesylation chemistry followed by displacement with sodium azide and hydrogenation over a catalyst such as Pd/C. The amine can be further acylated or alkylated, followed by the removal of the BOC group. The resulting amine can be coupled with a fatty acid of the formula D to afford compounds of the formula W.

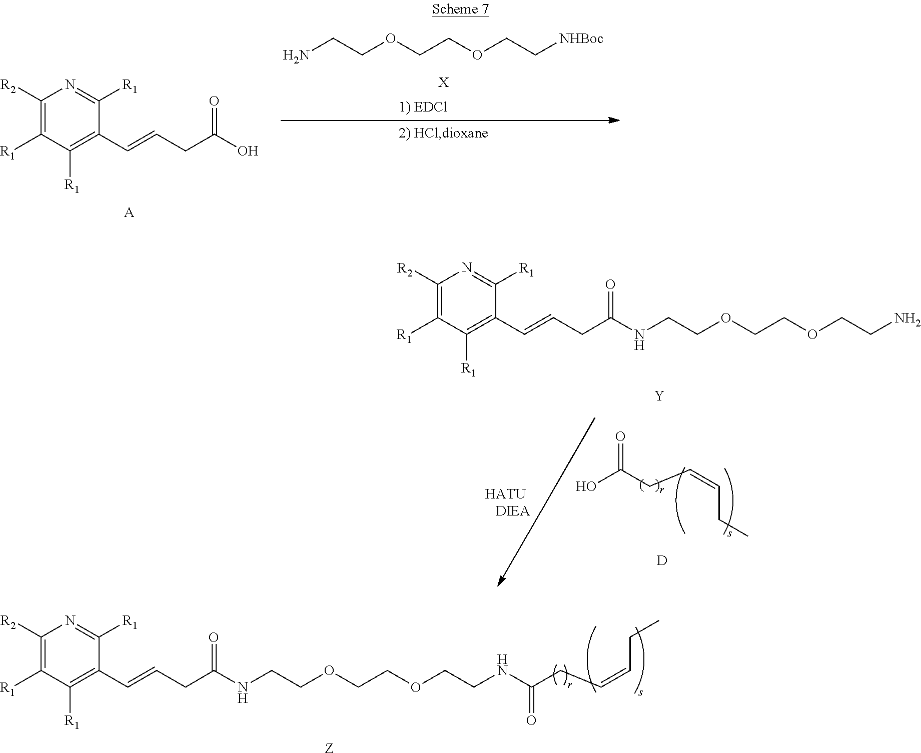

wherein R₁, R₂, r and s are as defined above.

Compound A can be amidated with the commercially available amine X using a coupling reagent such as DCC, CDI, EDC, optionally with a tertiary amine base and/or catalyst, e.g., DMAP to afford compound Y. The BOC group in compound Y can be removed with acids such as TFA or HCl in a solvent such as CH₂Cl₂ or dioxane. The resulting amine can be coupled with a fatty acid of the formula D using a coupling agent such as HATU in the presence of an amine such as DIEA to afford compounds of the formula Z.

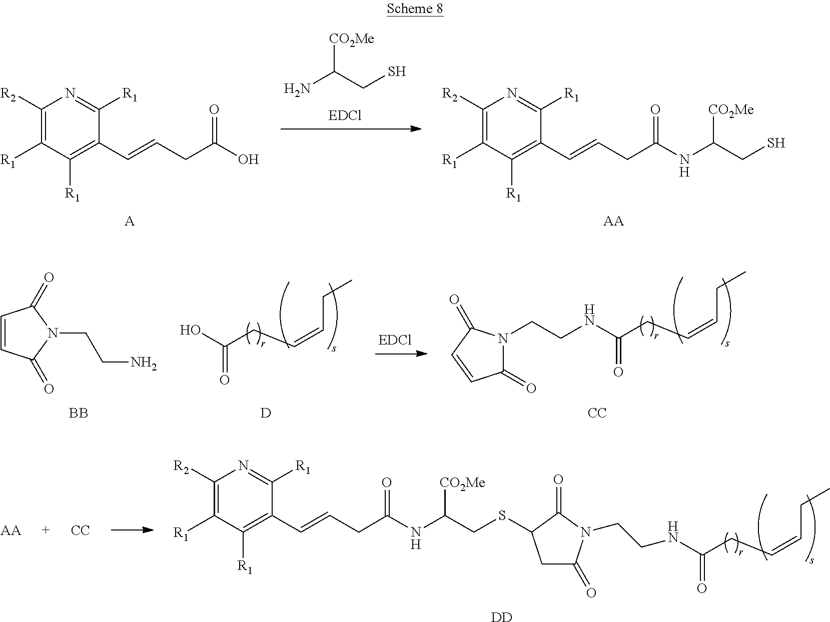

wherein R₁, R₂, r and s are as defined above.

Compound A can be amidated with the commercially available cysteine methyl ester using a coupling reagent such as DCC, CDI, EDC, or optionally with a tertiary amine base and/or catalyst, e.g., DMAP, to afford compound AA. The commercially available maleimide derivative BB can be coupled with a fatty acid of the formula D using a coupling agent such as HATU or EDCI to afford compounds of the formula CC. Compound AA can be coupled to compounds of the formula CC in a solvent such as acetonitrile to afford compounds of the formula DD.

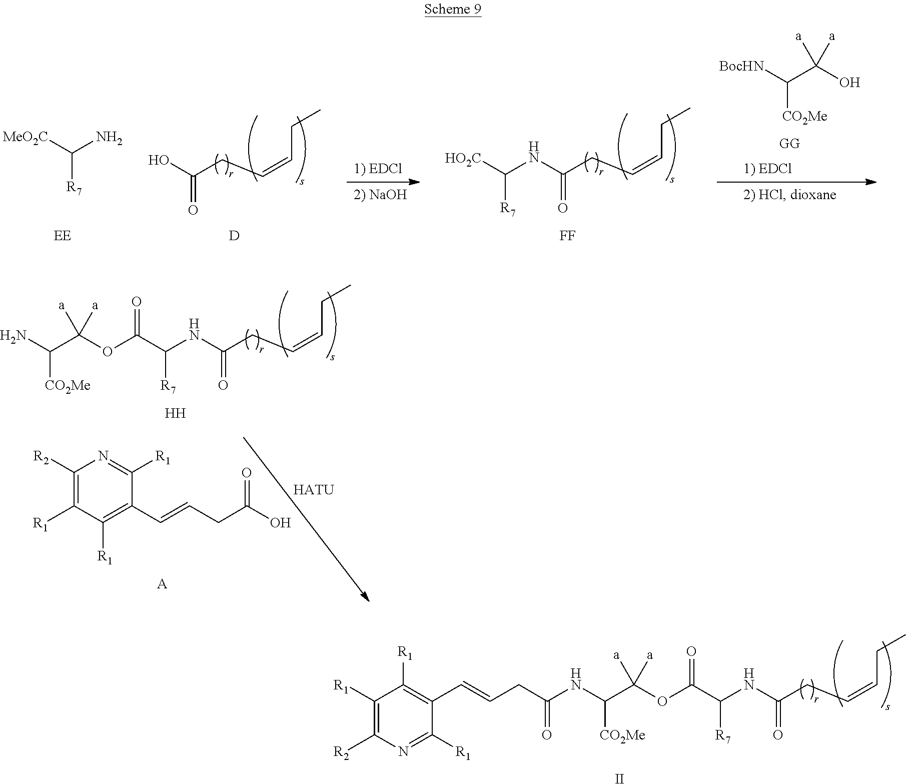

wherein R₁, R₂, R₇, a, r, and s are as defined above.

The commercially available amino acid esters EE can be coupled with a fatty acid of the formula D using a coupling agent such as EDCI or HATU, followed by alkaline hydrolysis of the methyl ester to afford compounds of the formula FF. Compounds of the formula FF can be coupled with the commercially available BOC-amino acid derivatives GG using a coupling agent such as EDCI or HATU. The BOC group can be removed by treatment with acids such as TFA or HCl to afford compounds of the formula HH which can then be coupled with compound

A to afford compounds of the formula II.

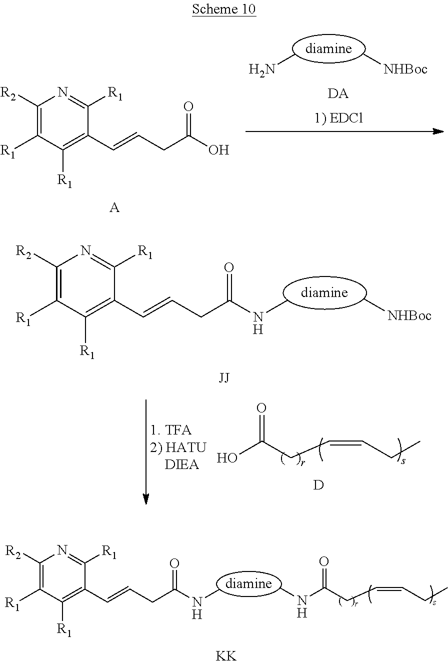

An acid of formula A can be coupled with a BOC-protected diamine of the general formula DA to obtain the BOC-protected amide derivative. After treatment with HCl in dioxane, the resulting amine can be coupled with a fatty acid of the formula D in order to obtain compounds of the formula KK. A variety of BOC-protected diamines are commercially available. The following diamines can be prepared according to the procedures outlined in the corresponding references:

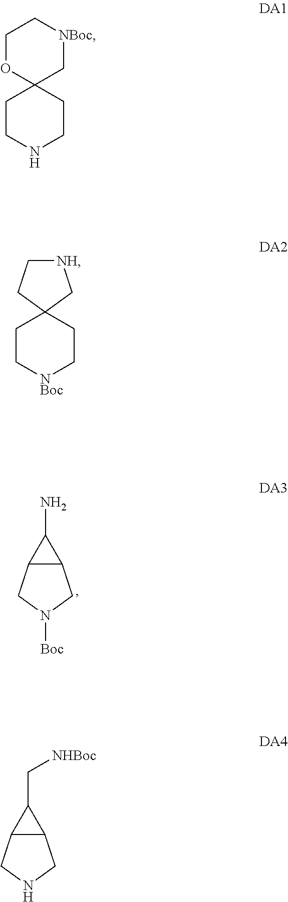

diamine DA1, Stocks et al, Bioorganic and Medicinal Chemistry Letters 2010, p. 7458; diamine DA2, Fritch et al, Bioorganic and Medicinal Chemistry Letters 2010, p. 6375; diamine DA3 and DA4, Moffat et al, J. Med. Chem. 2010, 53, p. 8663-86′78). To those familiar in the art, detailed procedures to prepare a variety of mono-protected diamines can also be found in the following references: WO 2004092172, WO 2004092171, and WO 2004092173.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effect of Fatty Acid Niacin Derivatives on ApoB Secretion in HepG2 Cells

Niacin has been reported to increase serum levels of HDL to LDL cholesterol in vivo. Similarly, niacin has been reported to increase the secretion of ApoA1 (Jin, F-Y. et al. Arterioscler. Thromb. Vasc. Biol. 1997, 17 (10), 2020-2028) while inhibiting the secretion of ApoB (Jin, F-Y. et al. Arterioscler. Thromb. Vasc. Biol. 1999, 19, 1051-1059) in the media supernatants of HepG2 cultures. Independently, DHA has been demonstrated to lower ApoB as well (Pan, M. et al. J. Clin. Invest. 2004, 113, 1277-1287) by a very different mechanism. Thus, the secretion of ApoB from HepG2 cells possesses utility as a cell based read-out for fatty acid non-flushing niacin derivatives.

HepG2 cells (ATCC) are seeded at 10,000 cells per well in 96 well plates. After adhering overnight, growth media (10% FBS in DMEM) is removed and cells are serum starved for 24 hours in DMEM containing 0.1% fatty acid free bovine serum albumin (BSA, Sigma). Cells are then treated with the compounds of the invention. Niacin at 5 mM can be used as a positive control. All treatments are performed in triplicate. Simultaneous with compound treatment, ApoB secretion is stimulated with addition of 0.1 oleate complexed to fatty acid free BSA in a 5:1 molar ratio. Incubation with compounds and oleate is conducted for 24 hours. Media supernatants are removed and ApoB concentrations are measured using ELISA kits (Mabtech AB). Percent inhibition of ApoB secretion is determined by normalizing data to vehicle treated wells. For a given compound, an IC₅₀ (concentration at which 50% of ApoB secretion is inhibited) can also be determined by using a 4 parameter-fit inhibition curve model (Graph Pad Prism®). In each experiment, cell viability is determined using the ATPlite 1-Step kit (Perkin Elmer), such that compound effects due to cytotoxicity can be monitored.

### Example 2

### Effect of Fatty Acid Niacin Conjugates on SREBP-1c Target Genes HepG2 cells (ATCC) are seeded at 20,000 cells per well in 96 well plates. After adhering overnight, growth media (10% FBS in DMEM) is removed and cells are serum starved for 24 hours in DMEM containing 1% fatty acid free bovine serum albumin (BSA, Sigma). Cells are then treated with the compounds of the invention at a final concentration of 50 μM in 1% BSA or 0.1 oleate complexed to fatty acid free BSA in a 5:1 molar ratio (the four compounds were compound I-7, compound I-8, a combination of niacin and DHA, a combination of niacin and EPA). Cells are incubated for 6 hours and then washed with PBS. RNA is reverse-transcribed using the cells II cDNA reagents according to standard protocols (outlined in Applied Biosystem StepOne Real-time PCR protocols). Real time PCR of transcripts is performed with Tagman assays for the three specific genes FASN (fatty acid synthase), SCD (steroyl CoA desaturase) and ApoA1 (apolipoprotein A1). In all three cases, 185-VICO is used as a normalization control.

### Compounds

The following non-limiting compound examples serve to illustrate further embodiments of the fatty acid non-flushing niacin derivatives. It is to be understood that any embodiments listed in the Examples section are embodiments of the fatty acid non-flushing niacin derivatives and, as such, are suitable for use in the methods and compositions described above.

### Example 3

### Preparation of (4Z,7Z,10Z,13Z,16Z,19Z)-N-(2-((E)-4-(pyridin-3-yl)but-3-enamido)ethyl)docosa-4,7,10,13,16,19-hexaenamide (I-1)

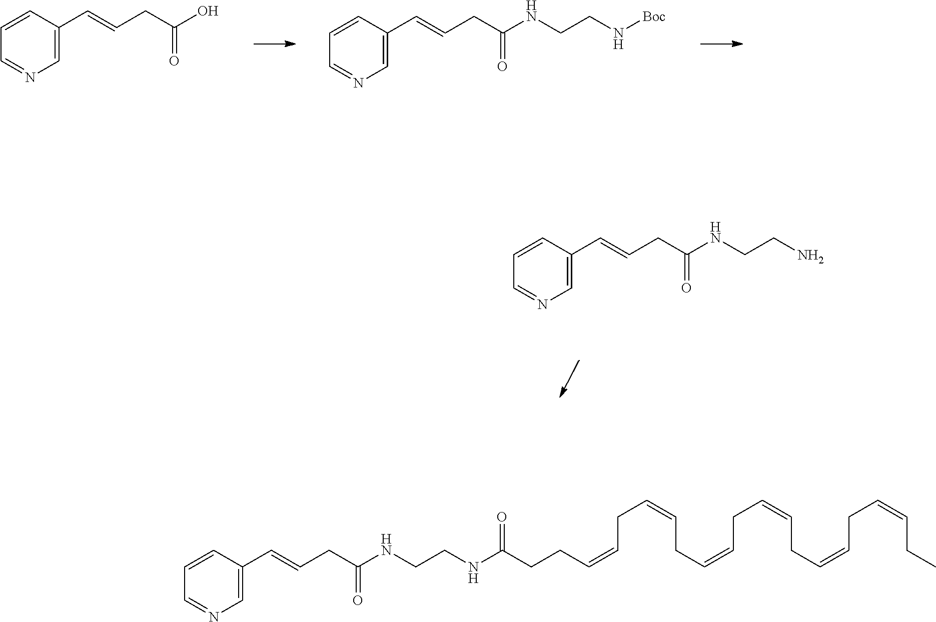

(E)-4-(Pyridin-3-yl)but-3-enoic acid can be prepared according to the procedure outlined in WO 2008/016968. In a typical run, (E)-4-(pyridin-3-yl)but-3-enoic acid (15 mmol) is taken up in CH₂Cl₂ (20 mL) along with oxalyl chloride (5 mmol). After a few drops of DMF are added, the reaction mixture is stirred at room temperature until all the solids has dissolved and all gas evolution has ceased (1 h). This freshly prepared solution of the acid chloride is added dropwise at 0° C. to a solution containing tert-butyl 2-aminoethylcarbamate (15 mmol) and Et₃N (23 mmol) in CH₂Cl₂ (200 mL). The resulting reaction mixture is warmed to room temperature and stirred for 2 h. It is then washed with brine, dried over Na₂SO₄, filtered and concentrated under reduced pressure. The resulting residue can be purified by silica gel chromatography (CH₂Cl₂) to afford (E)-tert-butyl 2-(4-(pyridin-3-yl)but-3-enamido)ethylcarbamate.

(E)-tert-Butyl 2-(4-(pyridin-3-yl)but-3-enamido)ethylcarbamate (10 mmol) is taken up in 4 N HCl in dioxane (40 mL). The resulting reaction mixture is allowed to stir at room temperature for 6 h. The resulting solids are collected by filtration and dried to afford of the HCl salt of (E)-N-(2-aminoethyl)-4-(pyridin-3-yl)but-3-enamide.

The HCl salt of (E)-N-(2-aminoethyl)-4-(pyridin-3-yl)but-3-enamide (5.0 mmol) is taken up in CH₃CN (20 mL) along with (4Z,7Z,10Z,13Z,16Z,19Z)-docosa-4,7,10,13,16,19-hexaenoic acid (5.0 mmol), HATU (5.5 mmol) and DIEA (15 mmol). The resulting reaction mixture is stirred at room temperature for 4 h and diluted with EtOAc. The organic layer is washed with saturated aqueous NaHCO₃, brine, dried over Na₂SO₄, filtered and concentrated under reduced pressure. The resulting residue is purified by silica gel chromatography (5% MeOH—CH₂Cl₂) to afford (4Z,7Z,10Z,13Z,16Z,19Z)-N-(2-((E)-4-(pyridin-3-yl)but-3-enamido)ethyl)docosa-4,7,10,13,16,19-hexaenamide.

### Example 4

### Preparation of (5Z,8Z,11Z,14Z,17Z)-N-(2-((E)-4-(pyridin-3-yl)but-3-enamido)ethyl)icosa-5,8,11,14,17-pentaenamide (I-2)

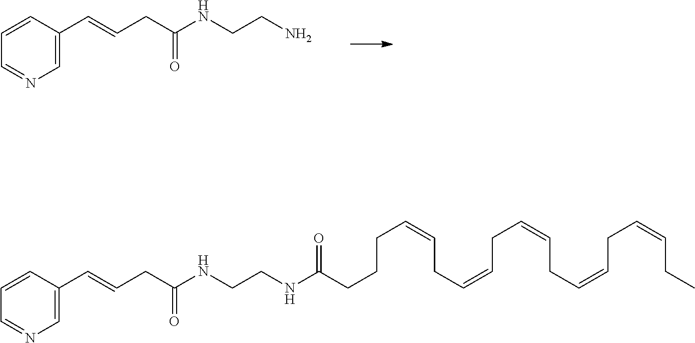

The HCl salt of (E)-N-(2-aminoethyl)-4-(pyridin-3-yl)but-3-enamide (5 mmol) is taken up in CH₃CN (15 mL) along with (5Z,8Z,11Z,14Z,17Z)-eicosa-5,8,11,14,17-pentaenoic acid (5 mmol), HATU (2.4 g, 6.3 mmol) and DIEA (15 mmol). The resulting reaction mixture is stirred at room temperature for 2 h and diluted with EtOAc. The organic layer is washed with saturated aqueous NaHCO₃, brine, dried over Na₂SO₄, filtered and concentrated under reduced pressure. The resulting residue is purified by silica gel chromatography (5% MeOH—CH₂Cl₂) to afford (5Z,8Z,11Z,14Z,17Z)-N-(2-((E)-4-(pyridin-3-yl)but-3-enamido)ethyl)icosa-5,8,11,14,17-pentaenamide.

### Example 5

### Preparation of (5Z,8Z,11Z,14Z,17Z)-N-(2-(2-(2-((E)-4-(pyridin-3-yl)but-3-enamido)ethyl)disulfanyl)ethyl)icosa-5,8,11,14,17-pentaenamide (I-6)

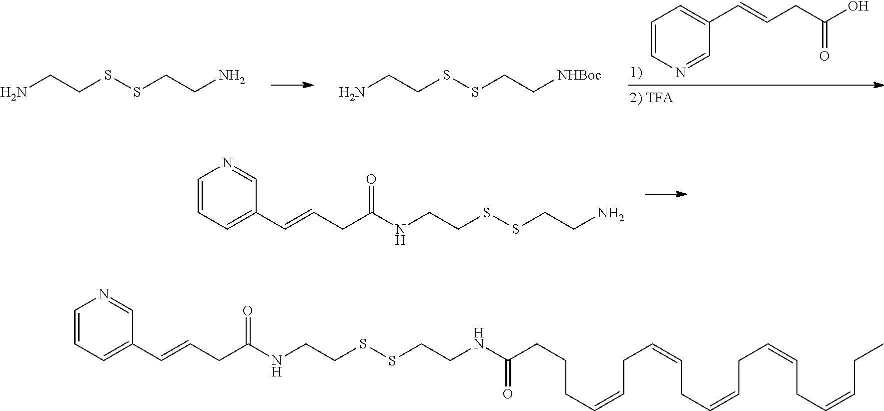

Cystamine dihydrochloride (1.0 g, 4.44 mmol) is dissolved in MeOH (50 mL). Triethylamine (1.85 mL, 3 eq) is added at room temperature, followed by dropwise addition of Boc₂O (0.97 g, 4.44 mmol) as a solution in MeOH (5 mL). The resulting reaction mixture is stirred at room temperature for 3 h. It is then concentrated under reduced pressure and the resulting residue is taken up in 1M aqueous NaH₂PO₄ (20 mL). The aqueous layer is washed with a 1:1 solution of pentane/EtOAc (10 mL), basified to pH 9 with 1M aqueous NaOH, and extracted with EtOAc. The combined organic layers are washed with brine, dried over Na₂SO₄, filtered and concentrated under reduced pressure to afford tert-butyl 2-(2-(2-aminoethyl)disulfanyl)ethylcarbamate.

Separately, (E)-4-(pyridin-3-yl)but-3-enoic acid (2.0 mmol) is taken up in CH₃CN (10 mL) along with tert-butyl 2-(2-(2-aminoethyl)disulfanyl)ethylcarbamate (2.0 mmol), and EDCI (2.2 mmol). The resulting reaction mixture is stirred at room temperature for 4 h and then diluted with EtOAc. The organic layer is washed with dilute aqueous NaHCO₃, brine, dried over Na₂SO₄, filtered and concentrated under reduced pressure. Purification by silica gel chromatography (CH₂Cl₂) affords (E)-tert-butyl 2-(2-(2-(4-(pyridin-3-yl)but-3-enamido)ethyl)disulfanyl)ethylcarbamate.

(E)-tert-Butyl 2-(2-(2-(4-(pyridin-3-yl)but-3-enamido)ethyl)disulfanyl)ethylcarbamate (0.5 mmol) is taken up in 25% TFA in CH₂Cl₂ solution (5 mL) and allowed to stir at room temperature for 4 h. The reaction mixture is then concentrated under reduced pressure to afford the TFA salt of (E)-N-(2-(2-(2-aminoethyl)disulfanyl)ethyl)-4-(pyridin-3-yl)but-3-enamide. This material is taken up in CH₃CN (10 mL) along with (5Z,8Z,11Z,14Z,17Z)-eicosa-5,8,11,14,17-pentaenoic acid (0.5 mmol), HATU (0.6 mmol) and DIEA (1.5 mmol). The resulting reaction mixture is stirred at room temperature for 2 h. It is then diluted with EtOAc and washed successively with saturated aqueous NaHCO₃ and brine. The organic layer is dried over Na₂SO₄, filtered and concentrated under reduced pressure. Purification by silica gel chromatography (5% MeOH—CH₂Cl₂) affords (5Z,8Z,11Z,14Z,17Z)-N-(2-(2-(2-((E)-4-(pyridin-3-yl)but-3-enamido)ethyl)disulfanyl)ethyl)icosa-5,8,11,14,17-pentaenamide.

### Example 6

### Preparation of (5Z,8Z,11Z,14Z,17Z)-N-(2-(2-((E)-4-(pyridin-3-yl)but-3-enamido)ethoxy)ethybicosa-5,8,11,14,17-pentaenamide (I-3)

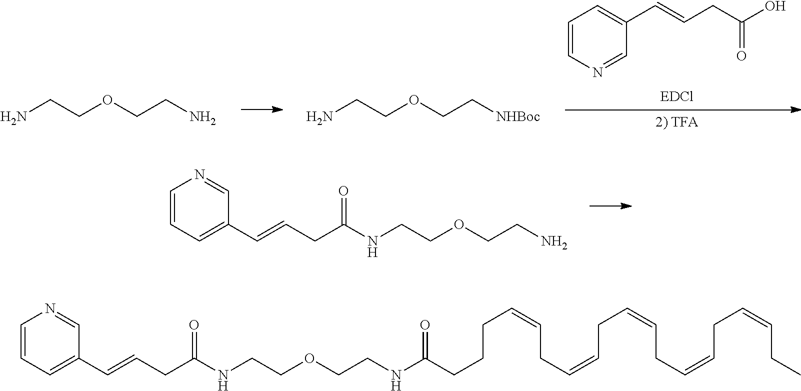

In a typical run, sodium hydroxide (400 mg, 10 mmol) is dissolved in MeOH (70 mL) and 2-(2-aminoethoxy)ethanamine dihydrochloride (1.0 g, 5.65 mmol) is added. The resulting reaction mixture is stirred at room temperature for 30 min. A solution containing Boc₂O (740 mg, 3.40 mmol) in THF (15 mL) is then added dropwise, at room temperature, over a period of 15 min. The resulting reaction mixture is stirred at room temperature for 18 h. It is then concentrated under reduced pressure. The resulting residue is taken up in CH₂Cl₂ (200 mL) and stirred vigorously at room temperature for 4 h. The mixture is filtered and the filtrate is concentrated under reduced pressure to afford tert-butyl 2-(2-aminoethoxy)ethylcarbamate.

tert-Butyl 2-(2-aminoethoxy)ethylcarbamate (2.0 mmol) is taken up in CH₃CN (20 mL) along with (E)-4-(pyridin-3-yl)but-3-enoic acid (2.0 mmol) and EDCI (2.2 mmol). The resulting reaction mixture is stirred at room temperature for 18 h. It is then diluted with EtOAc (20 mL), washed with saturated aqueous NaHCO₃, brine, dried over Na₂SO₄, filtered and concentrated under reduced pressure. The resulting residue is purified by silica gel chromatography (9:1 CH₂Cl₂/MeOH) to afford (E)-tert-butyl 2-(2-(4-(pyridin-3-yl)but-3-enamido)ethoxy)ethylcarbamate.

(E)-tert-Butyl 2-(2-(4-(pyridin-3-yl)but-3-enamido)ethoxy)ethylcarbamate (0.50 mmol) is taken up in 25% TFA in CH₂Cl₂ (10 mL). The reaction mixture is allowed to stir at room temperature for 2 h and then concentrated under reduced pressure to afford the TFA salt of (E)-N-(2-(2-aminoethoxy)ethyl)-4-(pyridin-3-yl)but-3-enamide. This material is then taken up in CH₃CN (10 mL) along with (5Z,8Z,11Z,14Z,17Z)-eicosa-5,8,11,14,17-pentaenoic acid (0.50 mmol), HATU (0.55 mmol) and DIEA (2 mmol). The resulting reaction mixture is stirred at room temperature for 2 h. It is then diluted with EtOAc and washed successively with saturated aqueous NaHCO₃ and brine. The organic layer is dried over Na₂SO₄, filtered and concentrated under reduced pressure. Purification by silica gel chromatography (9:1 CH₂Cl₂/MeOH) affords (5Z,8Z,11Z,14Z,17Z)-N-(2-(2-((E)-4-(pyridin-3-yl)but-3-enamido)ethoxy)ethyl)icosa-5,8,11,14,17-pentaenamide.

### Example 7

### Preparation of (5Z,8Z,11Z,14Z,17Z)-N-(2-(methyl(2-((E)-4-(pyridin-3-yl)but-3-enamido)ethyl)amino)ethyl)icosa-5,8,11,14,17-pentaenamide (I-5)

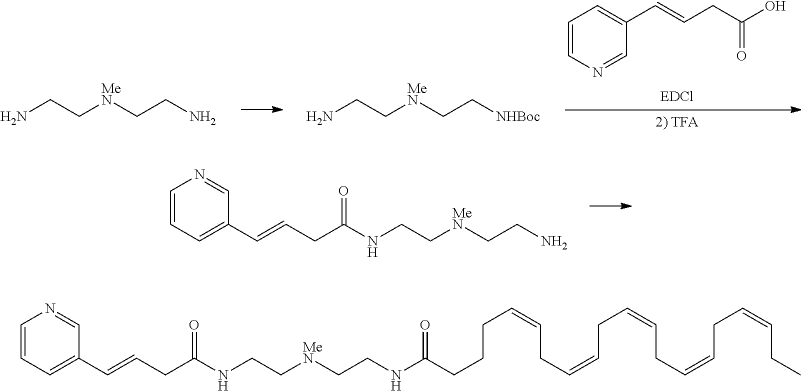

N1-(2-Aminoethyl)-N-1-methylethane-1,2-diamine (5.0 g, 42.7 mmol) is dissolved in CH₂Cl₂ (100 mL) and cooled to 0° C. A solution of Boc₂O (0.93 g, 4.27 mmol) in CH₂Cl₂ (10 mL) is then added dropwise at 0° C. over a period of 15 min. The resulting reaction mixture is stirred at 0° C. for 30 min and then warmed to room temperature. After stirring at room temperature for 2 h, the reaction mixture is diluted with CH₂Cl₂ (100 mL). The organic layer is washed with brine (3×25 mL), dried over Na₂SO₄, filtered and concentrated under reduced pressure to afford tert-butyl 2-((2-aminoethyl)(methyl)amino)ethylcarbamate.

tert-Butyl 2-((2-aminoethyl)(methyl)amino)ethylcarbamate (2 mmol) is taken up in CH₃CN (15 mL) along with (E)-4-(pyridin-3-yl)but-3-enoic acid (2 mmol) and EDCI (2.2 mmol). The resulting reaction mixture is stirred at room temperature for 18 h and then diluted with EtOAc. The organic layer is washed with saturated aqueous NaHCO₃, brine, dried over Na₂SO₄, filtered and concentrated under reduced pressure. The resulting residue is purified by silica gel chromatography (5% MeOH—CH₂Cl₂) to afford (E)-tert-butyl 2-(methyl(2-(4-(pyridin-3-yl)but-3-enamido)ethyl)amino)ethylcarbamate.

(E)-tert-butyl 2-(methyl(2-(4-(pyridin-3-yl)but-3-enamido)ethyl)amino)ethylcarbamate (0.25 mmol) is taken up in a 25% TFA in CH₂Cl₂ solution (5 mL) and allowed to stir at room temperature for 3 h. The reaction mixture is concentrated under reduced pressure to afford the TFA salt of (E)-N-(2-((2-aminoethyl)(methyl)amino)ethyl)-4-(pyridin-3-yl)but-3-enamide. This material is taken up in CH₃CN (10 mL) along with (5Z,8Z,11Z,14Z,17Z)-eicosa-5,8,11,14,17-pentaenoic acid (0.25 mmol), HATU (117 mg, 0.28 mmol) and DIEA (0.75 mmol). The resulting reaction mixture is stirred at room temperature for 2 h. It is then diluted with EtOAc and washed successively with saturated aqueous NaHCO₃ and brine. The organic layer is dried over Na₂SO₄, filtered and concentrated under reduced pressure. Purification by silica gel chromatography (5% MeOH—CH₂Cl₂) affords (5Z,8Z,11Z,14Z,17Z)-N-(2-(methyl(2-((E)-4-(pyridin-3-yl)but-3-enamido)ethyl)amino)ethyl)icosa-5,8,11,14,17-pentaenamide.

### Example 8

### Preparation of (S)-6-((5Z,8Z,11Z,14Z,17Z)-icosa-5,8,11,14,17-pentaenamido)-2-((E)-4-(pyridin-3-yl)but-3-enamido)hexanoic acid (I-7)

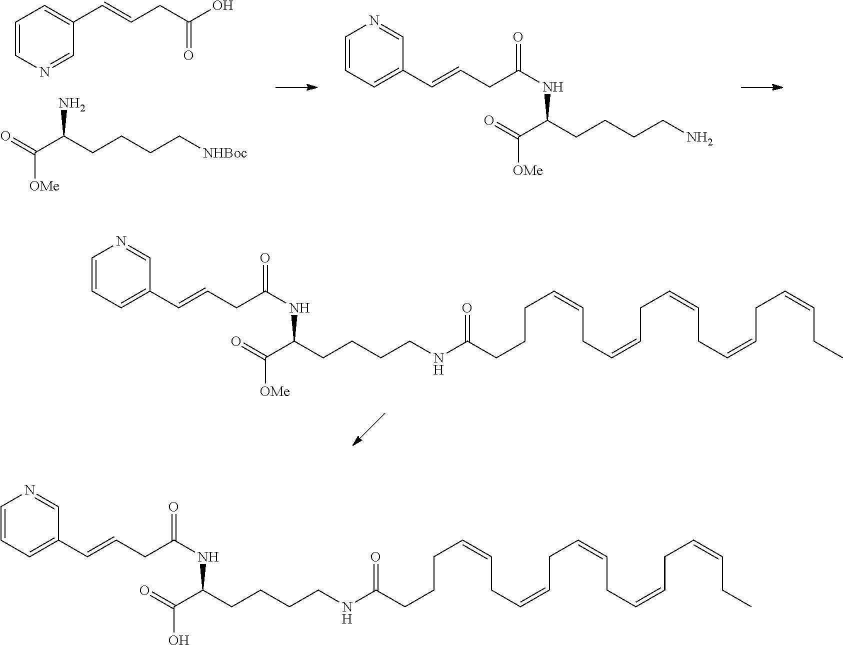

H-Lysine-(BOC)—OMe hydrochloride (2 mmol) is taken up in CH₃CN (10 mL) along with (E)-4-(pyridin-3-yl)but-3-enoic acid (2 mmol), EDCI (2.2 mmol) and DIEA (3 mmol). The resulting reaction mixture is stirred at room temperature for 18 h and diluted with EtOAc. The organic layer is washed with dilute aqueous NaHCO₃, brine, dried over Na₂SO₄, filtered and concentrated under reduced pressure. Purification by silica gel chromatography (CH₂Cl₂) affords (S,E)-methyl 6-(tert-butoxycarbonyl)-2-(4-(pyridin-3-yl)but-3-enamido)hexanoate. (S,E)-methyl 6-(tert-butoxycarbonyl)-2-(4-(pyridin-3-yl)but-3-enamido)hexanoate (0.7 mmol) is taken up in 4M HCl in dioxane (2 mL) and allowed to stir at room temperature for 1 h. The reaction mixture is diluted with EtOAc and concentrated under reduced pressure to afford the HCl salt of (S,E)-methyl 6-amino-2-(4-(pyridin-3-yl)but-3-enamido)hexanoate. This material is taken up in CH₃CN (5 mL) along with (0.7 mmol), HATU (0.77 mmol) and DIEA (2.2 mmol). The resulting reaction mixture is stirred at room temperature for 2 h and diluted with EtOAc. The organic layer is washed with dilute aqueous NaHCO₃, brine, dried over Na₂SO₄, filtered and concentrated under reduced pressure. Purification by silica gel chromatography (9:1 CH₂Cl₂/MeOH) affords (S)-methyl 6-((5Z,8Z,11Z,14Z,17Z)-icosa-5,8,11,14,17-pentaenamido)-2-((E)-4-(pyridin-3-yl)but-3-enamido)hexanoate.

(S)-Methyl 6-((5Z,8Z,11Z,14Z,17Z)-icosa-5,8,11,14,17-pentaenamido)-2-((E)-4-(pyridin-3-yl)but-3-enamido)hexanoate (0.07 mmol) is taken up in 2 mL of THF along with 80 μL of a 5 M NaOH solution. The resulting reaction mixture is stirred at room temperature for 2 h. It is then acidified to pH 4 with 2 N HCl and then extracted with EtOAc. The combined organic layers are dried (Na₂SO₄) and concentrated under reduced pressure to afford (S)-6-((5Z,8Z,11Z,14Z,17Z)-icosa-5,8,11,14,17-pentaenamido)-2-((E)-4-(pyridin-3-yl)but-3-enamido)hexanoic acid.

### Example 9

### Preparation of (S)-2-((5Z,8Z,11Z,14Z,17Z)-icosa-5,8,11,14,17-pentaenamido)-6-((E)-4-(pyridin-3-yl)but-3-enamido)hexanoic acid (I-8)

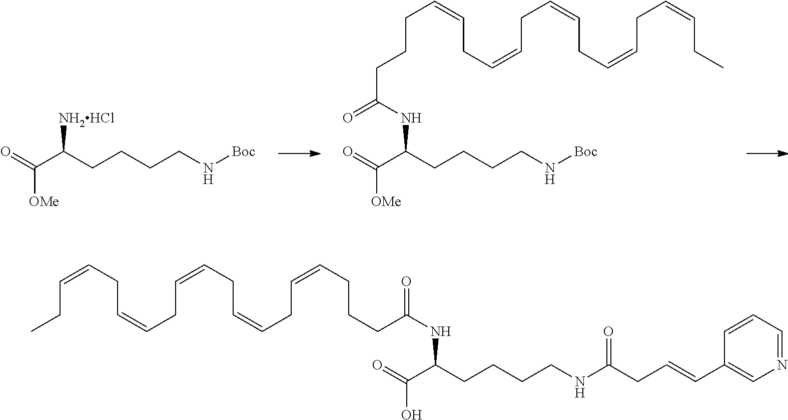

H-Lysine-(BOC)—OMe hydrochloride (2 mmol) is taken up in 30 mL of CH₃CN along with (5Z,8Z,11Z,14Z,17Z)-eicosa-5,8,11,14,17-pentaenoic acid (EPA, 2 mmol), HATU (2.2 mmol) and DIEA (6 mmol). The resulting reaction mixture is stirred at room temperature for 2 h. It is then diluted with EtOAc (70 mL) and washed with brine (20 mL). The organic layer is dried (Na₂SO₄) and concentrated under reduced pressure. The resulting residue is purified by silica gel chromatography (CH₂Cl₂, gradient elution to 90% CH₂Cl₂, 10% MeOH) to afford (S)-methyl 6-(tert-butoxycarbonyl)-2-((5Z,8Z,11Z,14Z,17Z)-icosa-5,8,11,14,17-pentaenamido)hexanoate.

(S)-methyl 6-(tert-butoxycarbonyl)-2-((5Z,8Z,11Z,14Z,17Z)-icosa-5,8,11,14,17-pentaenamido)hexanoate (1.0 mmol) is taken up in 4 mL of 4 N HCl in dioxane and allowed to stir at room temperature for 10 min. The reaction mixture is diluted with 10 mL of EtOAc and concentrated under reduced pressure to afford the HCl salt of (S)-methyl 6-amino-2-((5Z,8Z,11Z,14Z,17Z)-eicosa-5,8,11,14,17-pentaenamido)hexanoate. This residue is taken up in 5 mL of CH₃CN along with (E)-4-(pyridin-3-yl)but-3-enoic acid (1.0 mmol), HATU (1.1 mmol) and DIEA (3 mmol). The resulting reaction mixture is stirred at room temperature for 2 h and diluted with EtOAc (20 mL). The organic layer is washed with brine (20 mL), dried (Na₂SO₄) and concentrated under reduced pressure. The resulting residue was purified by chromatography (95% CH₂Cl₂, 5% MeOH) to afford (S)-methyl 2-((5Z,8Z,11Z,14Z,17Z)-icosa-5,8,11,14,17-pentaenamido)-6-((E)-4-(pyridin-3-yl)but-3-enamido)hexanoate.

((S)-methyl 2-((5Z,8Z,11Z,14Z,17Z)-icosa-5,8,11,14,17-pentaenamido)-6-((E)-4-(pyridin-3-yl)but-3-enamido)hexanoate (0.2 mmol) is taken up in 2 mL of THF along with an aqueous solution of NaOH (35 mg in 2 mL of H₂O). The resulting reaction mixture is stirred at room temperature for 2 h. It is then acidified to pH 4 with 2 N HCl and then extracted with EtOAc. The combined organic layers are dried (Na₂SO₄) and concentrated under reduced pressure to afford (S)-2-((5Z,8Z,11Z,14Z,17Z)-icosa-5,8,11,14,17-pentaenamido)-6-((E)-4-(pyridin-3-yl)but-3-enamido)hexanoic acid.

### Example 10

### Preparation of (5Z,8Z,11Z,14Z,17Z)-N-(2-((E)-4-(6-methylpyridin-3-yl)but-3-enamido)ethyl)icosa-5,8,11,14,17-pentaenamide (I-14)

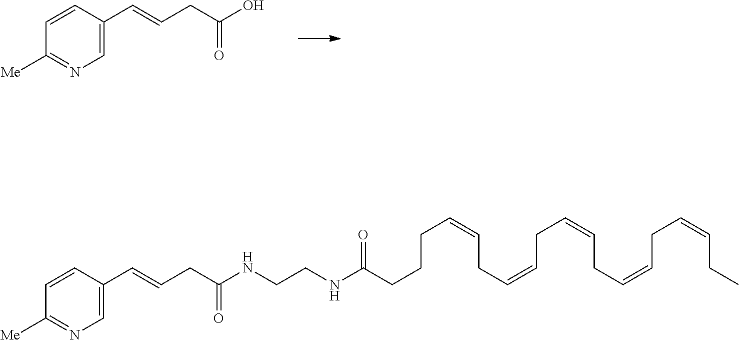

(E)-4-(6-Methylpyridin-3-yl)but-3-enoic acid can be prepared according to the procedures outlined in WO 2008/016968. This can be subjected to the same procedures outlined above to prepare (5Z,8Z,11Z,14Z,17Z)-N-(2-((E)-4-(6-methylpyridin-3-yl)but-3-enamido)ethyl)icosa-5,8,11,14,17-pentaenamide.

### Example 11

### Preparation of (5Z,8Z,11Z,14Z,17Z)-N-(2-((E)-4-(6-(2-(pyrrolidin-1-yl)ethyl)pyridin-3-yl)but-3-enamido)ethyl)icosa-5,8,11,14,17-pentaenamide (I-26)

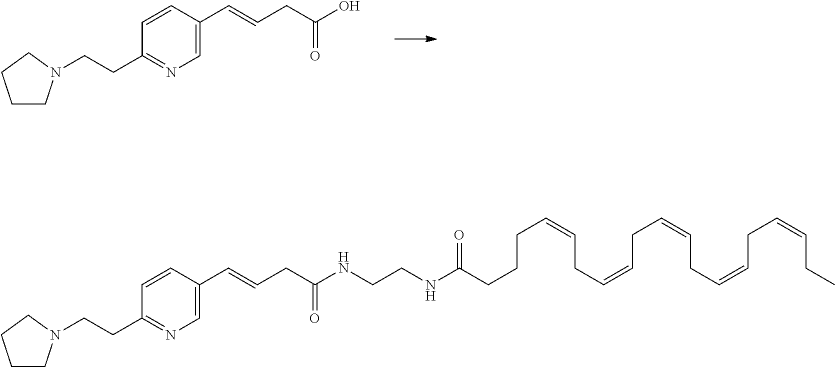

(E)-4-(6-(2-(Pyrrolidin-1-yl)ethyl)pyridin-3-yl)but-3-enoic acid can be prepared according to the procedures outlined in WO 2008/016968. This can be subjected to the same procedures outlined above to prepare (5Z,8Z,11Z,14Z,17Z)-N-(2-((E)-4-(6-(2-(pyrrolidin-1-yl)ethyl)pyridin-3-yl)but-3-enamido)ethyl)icosa-5,8,11,14,17-pentaenamide.

### Example 12

### Preparation of (R,E)-5-(1,2-dithiolan-3-yl)-N-(2-(4-(pyridin-3-yl)but-3-enamido)ethyl)pentanamide (I-37)

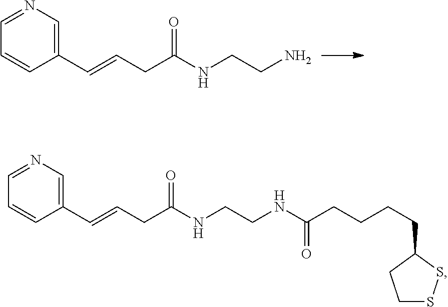

The HCl salt of (E)-N-(2-aminoethyl)-4-(pyridin-3-yl)but-3-enamide and R-lipoic acid can be subjected to the same reaction conditions outlined in example 3 to prepare (R,E)-5-(1,2-dithiolan-3-yl)-N-(2-(4-(pyridin-3-yl)but-3-enamido)ethyl)pentanamide.

### Example 13

### Preparation of N-(2-((4Z,7Z,10Z,13Z,16Z,19Z)-docosa-4,7,10,13,16,19-hexaenamido)ethyl)-6-(2-morpholinoethyl)nicotinamide (I-40)

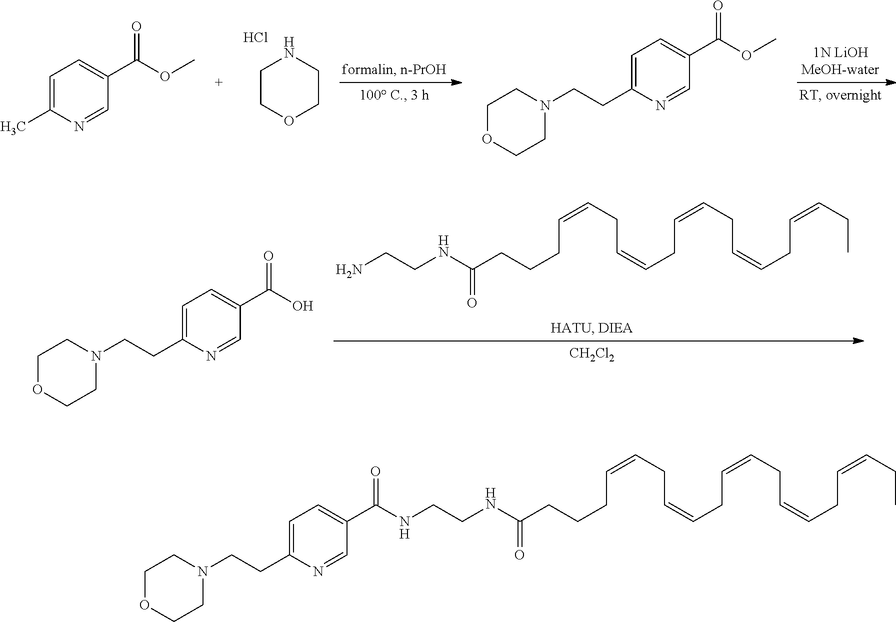

To a 50 mL round-bottom charged with stir bar, 1.67 g concentrated HCl (36% HCl) was slowly added to 1.44 g (16.5 mmol) morpholine and stirred for 10 minutes. In a 250 mL round-bottom charged with stir bar, 5 g of methyl 6-methylnicotinate (2.0 equivalents) was added to 21 mL n-PrOH, followed by addition of the morpholino-HCl mixture. Next, 1.32 g formalin (37%, in water, 1.0 equivalent) was added.

Reaction was stirred for 3 hours, under N₂ at 100° C. Upon completion, the reaction was dried under high vacuum, giving the crude morpholino-ester as a thick orange wax. The impurities were triturated from the crude product by re-dissolving in n-PrOH and adding diethyl ether until fine white precipitates form. The ether layer containing the white precipitate was decanted off. The trituration procedure was repeated twice more to give methyl 6-(2-morpholinoethyl)nicotinate as an orange wax without further purification.

To the methyl 6-(2-morpholinoethyl)nicotinic acid, was added 20 mL methanol and 10 mL water; the mixture was then subjected to an ice bath. Next, 15 mL 1N LiOH was added and reaction was allowed to reach room temperature, stirring overnight (16-18 hours). Upon completion, the reaction was adjusted to pH 2-3 with 5N HCl and dried under high vacuum to offer 6-(2-morpholinoethyl)nicotinic acid which was used in the final step without further purification. Yield: 31%. MS calculated for C₁₂H₁₆N₂O₃: 236.27. found 237.1 [M+H]⁺.

(5Z,8Z,11Z,14Z,17Z)-N-(2-Aminoethyl)icosa-5,8,11,14,17-pentaenamide was prepared according to the procedures outlined in WO 2011085211. To 710 mg (2.1 mmol) of (5Z,8Z,11Z,14Z,17Z)-N-(2-aminoethyl)icosa-5,8,11,14,17-pentaenamide in 5 mL CH₂Cl₂, was added 438 mg (0.9 equivalents) of 6-(2-morpholinoethyl)nicotinic acid. Next, HATU (958 mg, 1.2 equivalents), and 1 mL (3.0 equivalents) of diisopropylethylamine was added. The reaction was stirred at room temperature, under N₂ overnight. Upon completion, the reaction was washed with water, brine, dried (Na₂SO₄), and concentrated under high vacuum. The final product was purified by silica gel chromatography (95% CH₂Cl₂, 5% MeOH). MS calculated for C₃₄H₅₀N₄O₃: 562.79. found 607.3 [M−H+2Na]⁻ (negative ion mode).

### Example 14

### Preparation of N-(2-((5Z,8Z,11Z,14Z,17Z)-icosa-5,8,11,14,17-pentaenamido)ethyl)-6-(2-morpholinoethyl)nicotinamide (I-41)

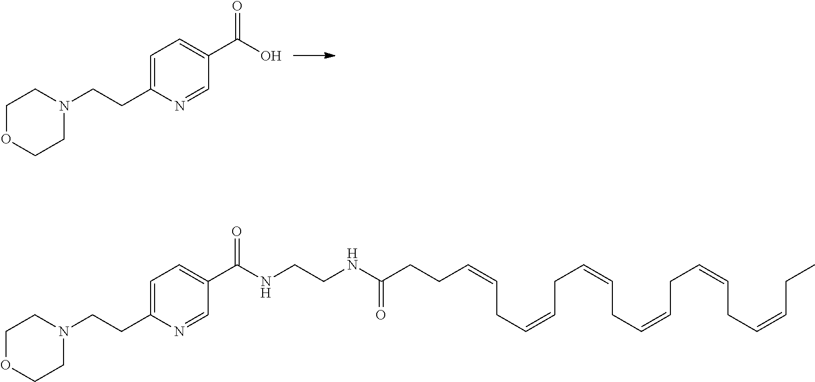

The synthesis of N-(2-((4Z,7Z,10Z,13Z,16Z,19Z)-docosa-4,7,10,13,16,19-hexaenamido)ethyl)-6-(2-morpholinoethyl)nicotinamide was carried out using the same procedure per synthesis of N-(2-((5Z,8Z,11Z,14Z,17Z)-icosa-5,8,11,14,17-pentaenamido)ethyl)-6-(2-morpholinoethyl)nicotinamide, substituting DHA for EPA. The final product was purified by silica gel chromatography (95% CH₂Cl₂, 5% MeOH). MS calculated for C₃₆H₅₂N₄O₃: 588.82. found 633.3 [M−H+2Na]⁻ (negative ion mode).

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising a non-flushing niacin and a fatty acid selected from lipoic acid, omega-3 fatty acids or fatty acids metabolized in vivo into omega-3 fatty acids.

2. A compound of Formula I: 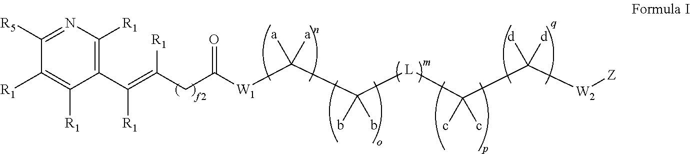
or a pharmaceutically acceptable salt, enantiomer or stereoisomer thereof;
wherein
Each R₁ and R₂ is independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;
R₅ is independently selected from the group consisting of H, -D, —Cl, —F, —CN, —NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₆ alkyl, —O—C₁-C₃ alkyl, —S(O)C₁-C₃ alkyl, —S(O)₂C₁-C₃ alkyl, an aryl, a cycloalkyl, a heterocycle and 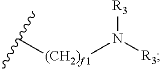
R₃ is independently H or C₁-C₆ alkyl, or both R₃ groups, when taken together with the nitrogen to which they are attached, can form 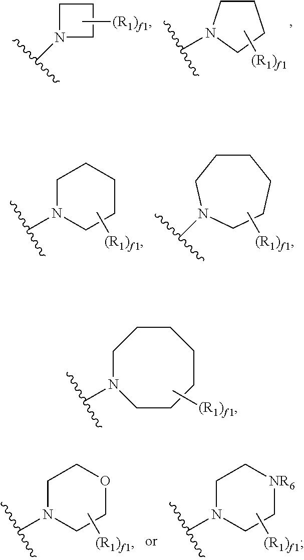
wherein f1 is 1, 2, 3 or 4; and f2 is 1, 2 or 3;
W₁ and W₂ are each independently null, O, S, NH, NR, or W₁ and W₂ can be taken together can form an imidazolidine or piperazine group, with the proviso that W₁ and W₂ can not be O simultaneously;
each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
each L is independently —O—, —S—, —S(O)—, —S(O)₂—, —S—S—, —(C₁-C₆alkyl)-, —(C₃-C₆cycloalkyl)-, a heterocycle, a heteroaryl, 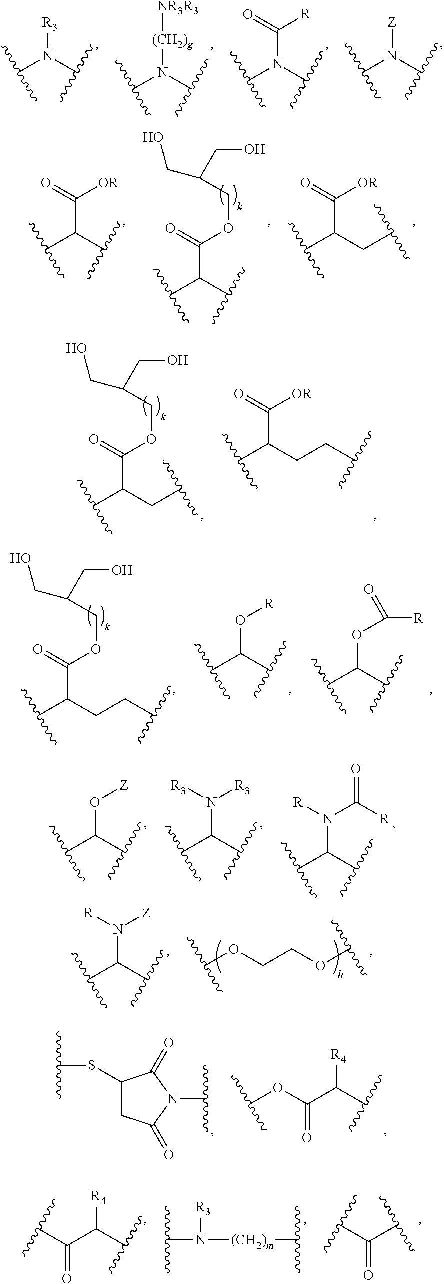 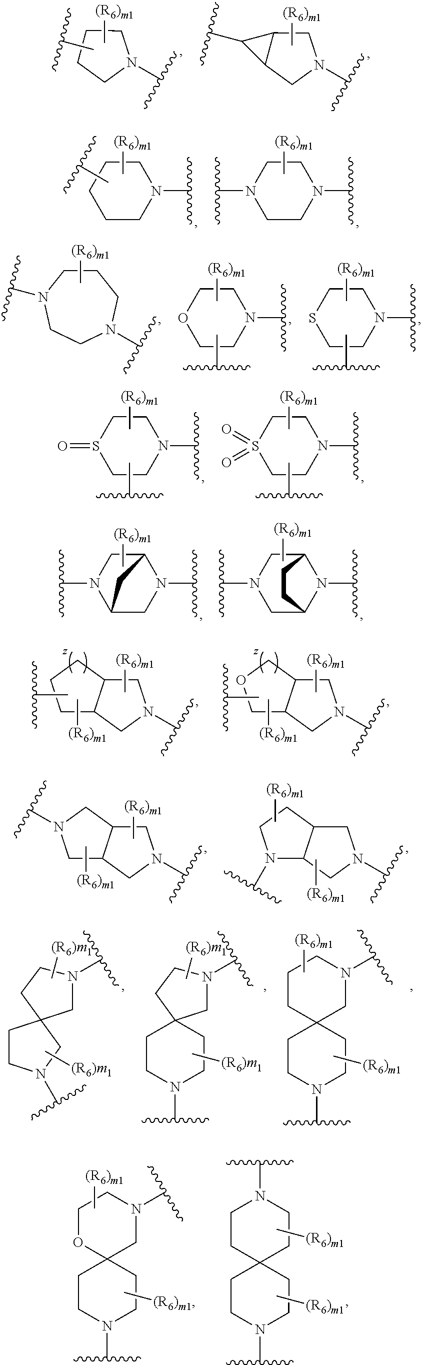 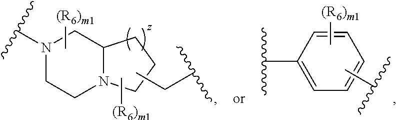
wherein the representation of L is not limited directionally left to right as is depicted, rather either the left side or the right side of L can be bound to the W₁ side of the compound of Formula I;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;
each g is independently 2, 3 or 4;
each h is independently 1, 2, 3 or 4;
m is 0, 1, 2, 3, 4 or 5; if m is more than 1, then L can be the same or different;
m1 is 0, 1, 2 or 3;
k is 0, 1, 2, or 3;
z is 1, 2, or 3;
each R₄ independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, or 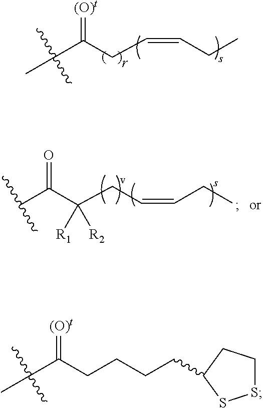
with the proviso that there is at least one 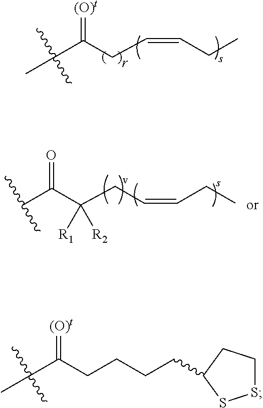
in the compound;
each r is independently 2, 3, or 7;
each s is independently 3, 5, or 6;
each t is independently 0 or 1;
each v is independently 1, 2, or 6;
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 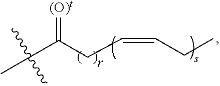
then t must be 0; and
when m, n, o, p, and q are each 0, and W₁ and W₂ are each null, then Z must not be

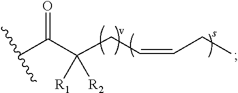

3. A compound of the Formula II: 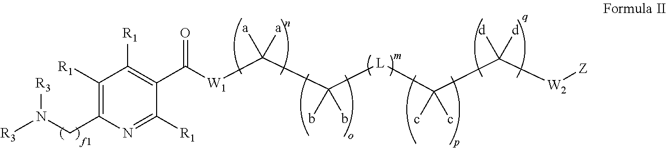
or a pharmaceutically acceptable salt, enantiomer or stereoisomer thereof;
wherein
each R₁ and R₂ is independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
R₃ is independently H or C₁-C₆ alkyl, or both R₃ groups, when taken together with the nitrogen to which they are attached, can form 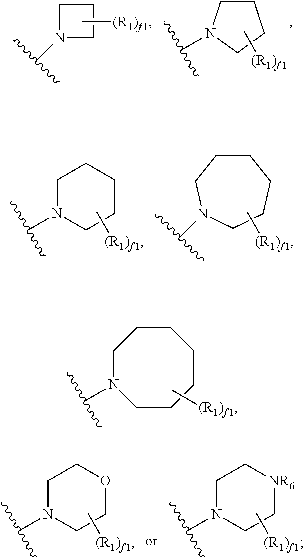
wherein f1 is 1, 2, 3 or 4;
W₁ and W₂ are each independently null, O, S, NH, NR, or W₁ and W₂ can be taken together can form an imidazolidine or piperazine group, with the proviso that W₁ and W₂ can not be O simultaneously;
each a, b, c, and d is independently —H, -D, —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
each L is independently-O—, —S—, —S(O)—, —S(O)₂—, —S—S—, —(C₁-C₆alkyl)-, —(C₃-C₆cycloalkyl)-, a heterocycle, a heteroaryl, 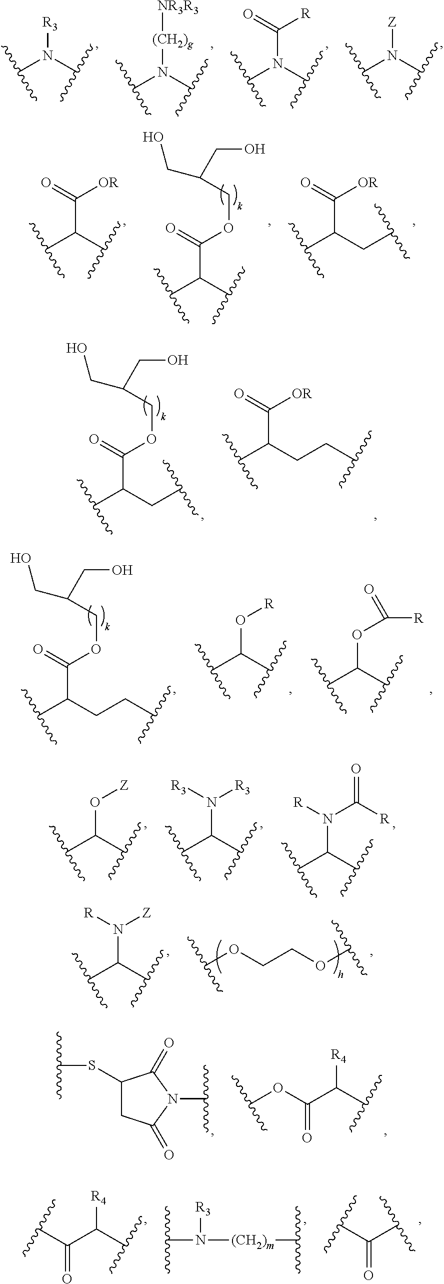 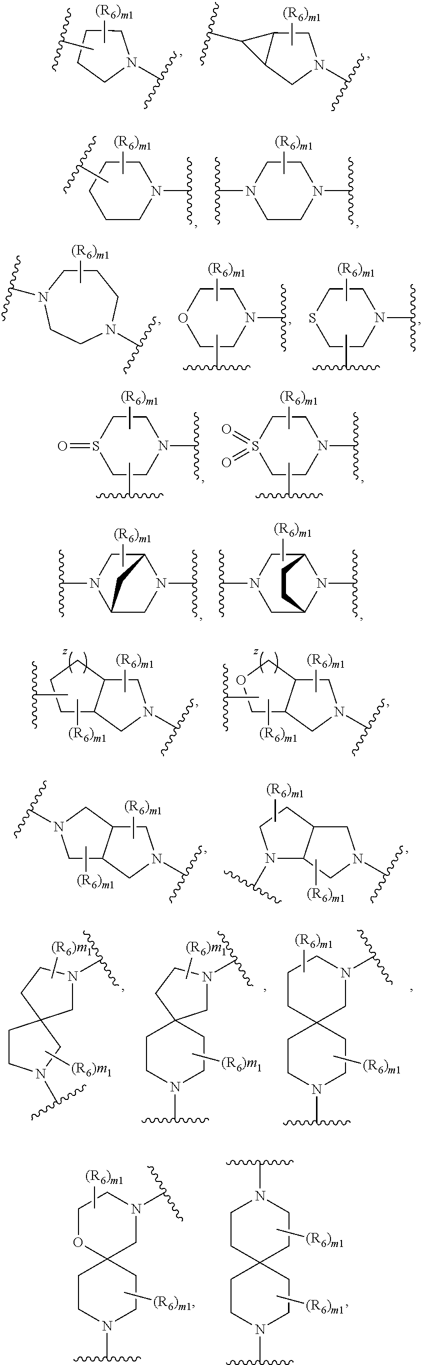 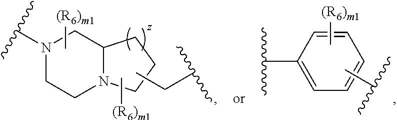
wherein the representation of L is not limited directionally left to right as is depicted, rather either the left side or the right side of L can be bound to the W₁ side of the compound of Formula II;
R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;
each g is independently 2, 3 or 4;
each h is independently 1, 2, 3 or 4;
m is 0, 1, 2, 3, 4 or 5; if m is more than 1, then L can be the same or different;
m1 is 0, 1, 2 or 3;
k is 0, 1, 2, or 3;
z is 1, 2, or 3;
each R₄ independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, or 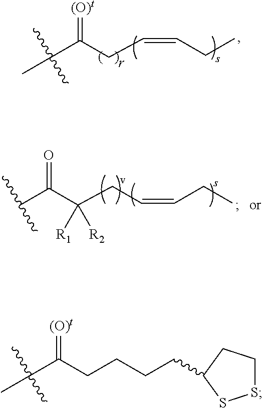
with the proviso that there is at least one 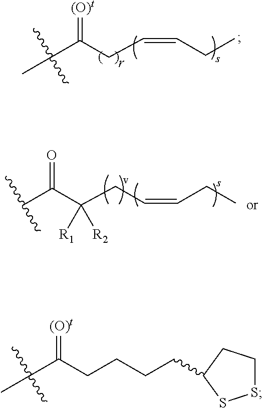
in the compound;
each r is independently 2, 3, or 7;
each s is independently 3, 5, or 6;
each t is independently 0 or 1;
each v is independently 1, 2, or 6;
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 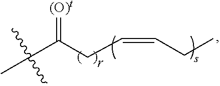
then t must be 0; and
when m, n, o, p, and q are each 0, and W₁ and W₂ are each null, then Z must not be

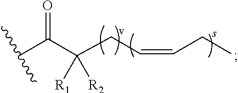

4. A pharmaceutical composition comprising a compound of claim 1 and a pharmaceutically acceptable carrier.

5. A method for treating a metabolic disease, the method comprising administering to a patient in need thereof an effective amount of a molecular conjugate of claim 1.

6. The method of claim 5, wherein the metabolic disease is selected from hypertriglyceridemia, hypercholesterolemia, fatty liver disease, nonalcoholic steatohepatitis (NASH), atherosclerosis, coronary heart disease, Type 2 diabetes, diabetic nephropathy, diabetic neuropathy, diabetic retinopathy, metabolic syndrome, or cardiovascular disease.

7. A pharmaceutical composition comprising a compound of claim 2 and a pharmaceutically acceptable carrier.

8. A method for treating a metabolic disease, the method comprising administering to a patient in need thereof an effective amount of a molecular conjugate of claim 2.

9. The method of claim 8, wherein the metabolic disease is selected from hypertriglyceridemia, hypercholesterolemia, fatty liver disease, nonalcoholic steatohepatitis (NASH), atherosclerosis, coronary heart disease, Type 2 diabetes, diabetic nephropathy, diabetic neuropathy, diabetic retinopathy, metabolic syndrome, or cardiovascular disease.

10. A pharmaceutical composition comprising a compound of claim 3 and a pharmaceutically acceptable carrier.

11. A method for treating a metabolic disease, the method comprising administering to a patient in need thereof an effective amount of a molecular conjugate of claim 3.

12. The method of claim 11, wherein the metabolic disease is selected from hypertriglyceridemia, hypercholesterolemia, fatty liver disease, nonalcoholic steatohepatitis (NASH), atherosclerosis, coronary heart disease, Type 2 diabetes, diabetic nephropathy, diabetic neuropathy, diabetic retinopathy, metabolic syndrome, or cardiovascular disease.
